(19) United States
(12) Patent Application Publication
(10) Pub. No.: US 2009/0068669 A1
(43) Pub. Date: Mar. 12, 2009

(54) SLC9A3R1 DIRECTED DIAGNOSTICS FOR NEOPLASTIC DISEASE

(76) Inventors: Elias Georges, Laval (CA); Claudia Boucher, Ile Perrot (CA); Julie Lanthier, Laval (CA); Anne-Marie Bonneau, Laval (CA)

Correspondence Address: WILMERHALE/BOSTON, 60 STATE STREET, BOSTON, MA, 02109, US

(21) Appl. No.: 12/209,839

(22) Filed: Sep. 12, 2008

Related U.S. Application Data
(60) Provisional application No. 60/993,572, filed on Sep. 12, 2007.

(51) Int. Cl. C12Q 1/68; G01N 33/566

(52) U.S. Cl. 435/6; 435/7.1

(57) ABSTRACT

Disclosed are methods for diagnosing cancer in a test cell sample or fluid sample by detecting an increase in the level of expression of SLC9A3R1 in the test cell sample or fluid sample as compared to the level of expression of SLC9A3R1 in a control cell sample or fluid sample isolated from a normal subject.

This application claims the benefit of priority to U.S. Provisional Application No. 60/993,572, filed Sep. 12, 2007, the specification of which is incorporated by reference in its entirety.

### FIELD OF THE INVENTION

The present invention relates generally to the field of medicine. More specifically, the invention pertains to methods and devices for detecting the development of cancer in a subject.

### BACKGROUND OF THE INVENTION

Cancer is one of the deadliest illnesses in the United States. It accounts for nearly 600,000 deaths annually in the United States, and costs billions of dollars for those who suffer from the disease. This disease is in fact a diverse group of disorders, which can originate in almost any tissue of the body. In addition, cancers may be generated by multiple mechanisms including pathogenic infections, mutations, and environmental insults (see, e.g., Pratt et al. (2005) Hum Pathol. 36(8): 861-70). The variety of cancer types and mechanisms of tumorigenesis add to the difficulty associated with treating a tumor, increasing the risk posed by the cancer to the patient's life and wellbeing.

Cancers manifest abnormal growth and the ability to move from an original site of growth to other tissues in the body (hereinafter termed “metastasis”), unlike most non-cancerous cells. These clinical manifestations are therefore used to diagnose cancer because they are applicable to all cancers. Additionally, a cancer diagnosis is made based on identifying cancer cells by their gross pathology through histological and microscopic inspection of the cells. Although the gross pathology of the cells can provide accurate diagnoses of the cells, the techniques used for such analysis are hampered by the time necessary to process the tissues and the skill of the technician analyzing the samples. These methodologies can lead to unnecessary delay in treating a growing tumor, thereby increasing the likelihood that a benign tumor will acquire metastatic characteristics. It is thus necessary to accurately diagnose potentially cancerous growths in early stages and as quickly as possible to avoid the development of a potentially life threatening illness.

One potential method of increasing the speed and accuracy of cancer diagnoses is the examination of genes as markers for neoplastic potential. Recent advances in molecular biology have identified genes involved in cell cycle control, apoptosis, and metabolic regulation (see, e.g., Isoldi et al. (2005) Mini Rev. Med. Chem. 5(7): 685-95). Mutations in many of these genes have also been shown to increase the likelihood that a normal cell will progress to a malignant state (see, e.g., Soejima et al. (2005) Biochem. Cell Biol. 83(4): 429-37). For example, mutations in p53, which is a well-known tumor suppressor gene, have been associated with aberrant cell growth leading to neoplastic potential (see Li et al. (2005) World J. Gastroenterol. 11(19): 2998-3001). Many mutations can affect the levels of expression of certain genes in the neoplastic cells as compared to normal cells.

There remains a need to identify an accurate and rapid means for diagnosing cancer in patients. Treatment efficacy would be improved by more efficient diagnoses of blood (including other bodily fluids) or tissue samples. Furthermore, rapid diagnoses of cancerous tissues or blood samples from patients would allow clinicians to treat potential tumors prior to the metastasis of the cancer to other tissues of the body. Finally, a test that did not rely upon a particular technician's skill at identifying abnormal histological characteristics would improve the reliability of cancer diagnoses. There is, therefore, a need for new methods of diagnoses for cancer that are accurate, fast, and relatively easy to interpret. In addition, such methods can be used to follow the response of patients to cancer treatment.

### SUMMARY OF THE INVENTION

The subject matter disclosed herein is based, in part, upon the discovery that differential expression of Solute carrier family 9 (sodium/hydrogen exchanger), member 3 regulator 1 (“SLC9A3R1”) at the protein and RNA levels occurs when a cell progresses to a neoplastic state. These expression patterns are therefore diagnostic for the presence of cancer in a cell sample. This discovery has been exploited to provide methods and compositions that use such patterns of expression to diagnose the presence of neoplastic cells in the test sample (cell sample or blood sample, where the protein is secreted or released in circulation). In addition the test sample may be bodily fluids, other than blood where the SLC9A3R1 is found as full length protein and/or peptides or fragments of SLC9A3R1. Similarly, a test sample may be blood or other bodily fluids containing the SLC9A3R1 RNA or modified nucleotide fragments of this gene.

Accordingly, in one aspect, a method of detecting a neoplasm is provided. The method comprises obtaining a potentially neoplastic test sample (blood or cells) and a control sample (blood or cells). The test sample can be obtained from a subject or a patient suffering from a neoplasm. The method includes detecting a level of expression of SLC9A3R1 in the test sample, and detecting the level of expression of SLC9A3R1 in the control sample. The method further includes comparing the level of expression of SLC9A3R1 in the test sample and comparing it to the level of expression of SLC9A3R1 in the control sample. The test sample is neoplastic or the patient harbors malignant tumor(s) if the level of expression of SLC9A3R1 in the test sample is greater than the level of expression of SLC9A3R1 in the control sample.

In some embodiments, the method includes isolating cytoplasmic fractions from the test cell sample and the control cell sample, and then separately detecting the levels of expression of SLC9A3R1 in the cytoplasmic fractions. In other embodiments, the level of expression of SLC9A3R1 protein is detected by contacting the test cell sample and the control cell sample with a protein binding agent selected from the group consisting of anti-SLC9A3R1-specific antibodies, anti-SLC9A3R1-specific fragments thereof, aptamers, and SLC9A3R1 inhibitors. In other embodiments, the method comprises detecting the level of protein binding agents bound to SLC9A3R1 protein by detecting a detectable label such as immunofluorescent label, radiolabel, and chemiluminescent label.

In some embodiments, the protein-binding agent is immobilized on a solid support. In other embodiments, the level of expression of SLC9A3R1 RNA is detected by contacting the test fluid and the non-neoplastic ovarian, lung, breast, prostate, colon control fluid with a nucleic acid binding agent such as RNA, cDNA, cRNA, or RNA-DNA hybrids. In certain embodiments, the level of nucleic acid binding agent hybridized to SLC9A3R1 RNA is detected by a detectable label operably linked to the binding agent such as an immunofluorescent label, a radiolabel, or a chemiluminescent label. In still other embodiments, the nucleic acid binding agent is immobilized on a solid support.

In some embodiments, the level of expression of anti-SLC9A3R1 antibody is detected in a test fluid sample and a control fluid sample. In certain embodiments, the level of expression of anti-SLC9A3R1 antibody is detected in a serum sample isolated from a subject. In certain other embodiments, the level of expression of anti-SLC9A3R1 antibody is detected using antibodies or fragments thereof. In particular embodiments, the antibodies or fragments thereof are operably linked to a detectable label such as an immunofluorescent label, radiolabel, and/or chemiluminescent label.

In some embodiments, the level of expression of SLC9A3R1 in the test fluid sample is at least 1.5 times greater than the level of expression of SLC9A3R1 in the control fluid sample. In other embodiments, the level of expression of SLC9A3R1 in the test fluid sample is at least 2 times greater than the level of expression of SLC9A3R1 in the control fluid sample. In still other embodiments, the level of expression of SLC9A3R1 in the test fluid sample is at least 4 times greater than the level of expression of SLC9A3R1 in the control fluid sample. In alternative embodiments, the level of expression of SLC9A3R1 in the test fluid sample is at least 6 times greater than the level of expression of SLC9A3R1 in the control fluid sample. In other embodiments, the level of expression of SLC9A3R1 in the test fluid sample is at least 8 times greater than the level of expression of SLC9A3R1 in the control fluid sample. In certain embodiments, the level of expression of SLC9A3R1 in the test fluid sample is at least 10 times greater than the level of expression of SLC9A3R1 in the control fluid sample. In some embodiments, the level of expression of SLC9A3R1 in the test fluid sample is at least 20 times greater than the level of expression of SLC9A3R1 in the control fluid sample.

In some embodiments, the test cell sample is obtained from a patient suffering from a metastasized ovarian neoplastic disease isolated from a tissue such as blood, bone marrow, spleen, lymph node, liver, thymus, kidney, brain, skin, gastrointestinal tract, eye, breast, and prostate. In other embodiments, the test cell sample is obtained from a patient suffering from an ovarian neoplasm such as ovarian carcinoma, ovarian epithelial adenocarcinoma, ovarian adenocarcinoma, sex cord-stromal carcinoma, endometrioid tumors, mucinous carcinoma, germ cell tumors, and clear cell tumors.

In some embodiments, the test cell sample is obtained from a patient suffering from a metastasized breast neoplastic disease isolated from a tissue such as blood, bone marrow, spleen, lymph node, lung, liver, bone, or brain, or in lymph nodes. In other embodiments, the test cell sample is obtained from a patient suffering from a breast neoplasm such as ductal or lobular carcinomas, ductal carcinoma in situ or invasive carcinoma.

In some embodiments, the test cell sample is obtained from a patient suffering from a metastasized lung neoplastic disease isolated from a tissue such as blood, bone marrow, spleen, lymph node, liver, bone, or brain, or in lymph nodes. In other embodiments, the test cell sample is obtained from a patient suffering from lung neoplasm such as Mesothelioma, small cell lung cancer, and Non-small cell lung cancer (i.e., Squamous cell carcinoma, Adenocarcinoma, and Large cell carcinoma).

In some embodiments, the test cell sample is obtained from a patient suffering from a metastasized colon neoplastic disease isolated from a tissue such as blood, bone marrow, spleen, lymph node, liver, bone, or brain, or in lymph nodes. In other embodiments, the test cell sample is obtained from a patient suffering from colon adinocarcinomas, such as leiomyosarcoma, lymphoma, melanoma, and neuroendocrine tumors.

In some embodiments, the test cell sample is obtained from a patient suffering from a metastasized prostate neoplastic disease isolated from a tissue such as blood, bone marrow, spleen, lymph node, liver, bone, or brain, or in lymph nodes. In other embodiments, the test cell sample is obtained from a patient suffering from prostate carcinomas.

In some embodiments, the probe for detecting SLC9A3R1 is an anti-SLC9A3R1 antibody or binding fragment thereof. In other embodiments, the probe for detecting SLC9A3R1 is an aptamer, SLC9A3R1 ligand, or SLC9A3R1-binding protein. In still other embodiments, the second probe is selected from the group consisting of a enolase 1 antibody, triosephosphate isomerase antibody, a cytokeratin 18 antibody, a stratifin (“SFN”) antibody, CRAB-PII antibody, hypoxanthine/guanine phosphoribosyltransferase (“HPRT”) antibody, and marker-specific binding fragments thereof, and combinations thereof. In certain embodiments, the second probe is selected from the group consisting of α enolase 1 ligand, triosephosphate isomerase ligand, a cytokeratin 18 ligand, a stratifin ligand, CRAB PII, hypoxanthine/guanine phosphoribosyltransferase ligand and combinations thereof. In some embodiments, the first probe detects SLC9A3R1 present in the test cell sample if the patient is suffering from neoplastic disease. In still other embodiments, the second probe detects a marker present on the surface of the test cell if the patient is suffering from neoplastic disease. In some embodiments, the first and second probes are immobilized on a solid support.

In still another aspect, a method of diagnosing cancer in a subject is provided. The method comprises the step of obtaining a test ovarian, breast, lung, colon or prostate fluid sample and a control fluid sample from a non-neoplastic ovarian, breast, lung, colon or prostate control sample. The method includes a step of detecting a level of expression of SLC9A3R1 in the test fluid sample, and detecting a level of expression of SLC9A3R1 in the control fluid sample. The method further includes a step of comparing the level of expression of SLC9A3R1 in the test fluid sample to the level of expression of SLC9A3R1 in the control fluid sample. Cancer is detected if the level of expression of SLC9A3R1 in the test fluid sample is greater than the level of expression of SLC9A3R1 in the control fluid sample.

In some embodiments, the method includes detecting the level of expression of SLC9A3R1 comprises isolating cellular cytoplasmic fractions from the test fluid sample and the control fluid sample, and separately detecting the level of expression of SLC9A3R1 in the cellular cytoplasmic fractions. In other embodiments, the level of expression of SLC9A3R1 protein is detected by contacting the test fluid sample and the control fluid sample with a protein binding agent selected from the group consisting of anti-SLC9A3R1 antibody and fragments thereof. In other embodiments, the level of protein binding agents bound to SLC9A3R1 protein is detected by a detectable label such as immunofluorescent label, radiolabel, and chemiluminescent label.

In some embodiments, the protein-binding agent is immobilized on a solid support. In other embodiments, the method involves the level of expression of SLC9A3R1 RNA is detected by contacting the test fluid and the non-neoplastic ovarian control fluid with a nucleic acid binding agent such as RNA, cDNA, cRNA, and RNA-DNA hybrids. In certain embodiments, the level of nucleic acid binding agent hybridized to SLC9A3R1 RNA is detected by a detectable label such as immunofluorescent label, radiolabel, and chemiluminescent label. In still other embodiments, the nucleic acid binding agent is immobilized on a solid support.

In some embodiments, the level of expression of SLC9A3R1 in the test fluid sample is 1.5 times greater than the level of expression of SLC9A3R1 in the control fluid sample. In other embodiments, the level of expression of SLC9A3R1 in the test fluid sample is 2 times greater than the level of expression of SLC9A3R1 in the control fluid sample. In still other embodiments, the level of expression of SLC9A3R1 in the test fluid sample is 4 times greater than the level of expression of SLC9A3R1 in the control fluid sample. In alternative embodiments, the level of expression of SLC9A3R1 in the test fluid sample is 6 times greater than the level of expression of SLC9A3R1 in the control fluid sample.

In other embodiments, the level of expression of SLC9A3R1 in the test fluid sample is 8 times greater than the level of expression of SLC9A3R1 in the control fluid sample. In certain embodiments, the level of expression of SLC9A3R1 in the test fluid sample is 10 times greater than the level of expression of SLC9A3R1 in the control fluid sample. In some embodiments, the level of expression of SLC9A3R1 in the test fluid sample is at least 20 times greater than the level of expression of SLC9A3R1 in the control fluid sample.

In some embodiments, the test fluid sample is from a patient suffering from a metastasized ovarian, breast, lung, colon or prostate neoplastic disease isolated from a tissue such as blood, bone marrow, spleen, lymph node, liver, thymus, kidney, brain, skin, saliva gastrointestinal tract, eye, breast, and prostate. In more embodiments, the test fluid sample is a patient suffering from an ovarian neoplasm such as ovarian carcinoma, ovarian epithelial adenocarcinoma, ovarian adenocarcinoma, sex cord-stromal carcinoma, endometrioid tumors, mucinous carcinoma, germ cell tumors, and clear cell tumors. In still other embodiments, the test cell sample is a cell such as blood cells, bone marrow cells, spleen cells, lymph node cells, liver cells, thymus cells, kidney cells, brain cells, skin cells, gastrointestinal tract cells, eye cells, breast cells, prostate cells, uterine cells, and ovary cells.

In some embodiments, the test cell sample is obtained from a patient suffering from a metastasized breast neoplastic disease isolated from a tissue such as blood, bone marrow, spleen, lymph node, lung, liver, bone, or brain, or in lymph nodes. In other embodiments, the test cell sample is obtained from a patient suffering from an breast neoplasm such as ductal or lobular carcinomas, ductal carcinoma in situ or invasive carcinoma.

In some embodiments, the test cell sample is obtained from a patient suffering from a metastasized lung neoplastic disease isolated from a tissue such as blood, bone marrow, spleen, lymph node, liver, bone, or brain, or in lymph nodes. In other embodiments, the test cell sample is obtained from a patient suffering from lung neoplasm such as Mesothelioma, small cell lung cancer, and Non-small cell lung cancer (e.g., Squamous cell carcinoma, Adenocarcinoma, and Large cell carcinoma).

In some embodiments, the test cell sample is obtained from a patient suffering from a metastasized colon neoplastic disease isolated from a tissue such as blood, bone marrow, spleen, lymph node, liver, bone, or brain, or in lymph nodes. In other embodiments, the test cell sample is obtained from a patient suffering from colon adinocarcinomas, such as leiomyosarcoma, lymphoma, melanoma, and neuroendocrine tumors.

In some embodiments, the test cell sample is obtained from a patient suffering from a metastasized prostate neoplastic disease isolated from a tissue such as blood, bone marrow, spleen, lymph node, liver, bone, or brain, or in lymph nodes. In other embodiments, the test cell sample is obtained from a patient suffering from prostate carcinomas.

In certain embodiments, the fluid sample is isolated from saliva, tears, urine, sweat, plasma, blood, or serum.

In another aspect, a kit for diagnosing or detecting neoplasia is provided. The kit includes a first probe for the detection of SLC9A3R1 and at least a second probe for the detection of a neoplasia marker such as enolase 1, triosephosphate isomerase, a cytokeratin 18, a stratifin, CRAB-PII, and HPRT.

In some embodiments, the probe for detecting SLC9A3R1 is an anti-SLC9A3R1 antibody or binding fragment thereof. In yet other embodiments, the probe is a ligand, aptamers, or inhibitor specific for SLC9A3R1. In still other embodiments, the second probe is selected from the group consisting of a enolase 1 antibody, triosephosphate isomerase antibody, a cytokeratin 18 antibody, a SFN antibody, CRAB-PII antibody, and HPRT antibody, and marker specific fragments thereof, and combinations thereof. In certain embodiments, the second probe includes a α enolase 1 ligand or aptamer, triosephosphate isomerase ligand or aptamer, a cytokeratin 18 ligand, a SFN ligand or aptamer, HPRT ligand, and CRAB-PII ligand or aptamers, and combinations thereof. In still other embodiments, the second probe detects a marker present of the surface of the test cell if the patient is suffering from ovarian neoplastic disease. In some embodiments, the first and second probes are immobilized on a solid support. In some embodiments, the SLC9A3R1 probe is a nucleic acid probe such as RNA, cDNA, cRNA, and RNA-DNA hybrids. In certain embodiments, the SLC9A3R1 probe is complementary to at least 20 a nucleotide sequence of a nucleic acid sequence consisting of SEQ ID NO: 1. In some embodiments, the second probe is a nucleic acid probe such as RNA, cDNA, cRNA, and RNA-DNA hybrids. In certain embodiments, the second probe is a nucleic acid probe complementary to at least a 20 nucleotide sequence of a nucleic acid sequence such as SEQ ID NOS: 2, 3, 4, 5, 6, and 7.

In other embodiments, the first probe binds to an anti-SLC9A3R1 antibody. In particular embodiments, the first probe is an antibody or fragment thereof operably linked to a detectable label.

In another aspect, a method for detecting a neoplasm is provided. The method entails obtaining a potentially neoplastic test sample and a non-neoplastic control sample and detecting a level of SLC9A3R1 expression in the test sample and in the control sample. The method further includes the step of detecting a level of expression of at least one of CRAB-PII, enolase I, cytokeratine 18, triosephosphate isomerase, SFN, and/or HPRT. The levels of expression of SLC9A3R1 and at least one of enolase I, cytokeratine 18, triosephosphate isomerase, SFN, and/or HPRT in the test sample are compared to the levels of expression of SLC9A3R1 and at least one of enolase I, cytokeratine 18, triosephosphate isomerase, SFN, and/or HPRT in the control sample. The test sample is neoplastic if the level of expression of SLC9A3R1 and at least one of enolase I, cytokeratine 18, triosephosphate isomerase, SFN, and/or HPRT in the test sample is greater than the level of expression of SLC9A3R1 and at least one of enolase I, cytokeratine 18, triosephosphate isomerase, SFN, and/or HPRT in the control sample.

In certain embodiments, the level of expression of SLC9A3R1 and the level of expression of at least one of enolase I, cytokeratine 18, triosephosphate isomerase, SFN, and/or HPRT is detected by isolating a cellular cytoplasmic fraction from the test sample and from the control sample, and then separately detecting the level of expression of SLC9A3R1 and at least one of enolase I, cytokeratine 18, triosephosphate isomerase, SFN, and/or HPRT in these cellular cytoplasmic fractions. In particular embodiments, the level of SLC9A3R1 expression is detected by contacting the test sample and the control sample with a SLC9A3R1-specific protein binding agent selected from the group consisting of an anti-SLC9A3R1-specific antibodies, anti-SLC9A3R1-specific fragments thereof, SLC9A3R1-specific ligands, SLC9A3R1-specific aptamers, and SLC9A3R1 inhibitors or SLC9A3R1-binding proteins.

In other embodiments, the protein binding agent is immobilized on a solid support. In still other embodiments, the level of expression of anti-SLC9A3R1 antibody is detected in a test cell or fluid sample and a control cell or fluid sample. In certain other embodiments, the level of expression of anti-SLC9A3R1 antibody is determined in a serum sample isolated from a subject and is also determined in a serum sample isolated from a subject not suffering from a neoplasm or cancer. In certain embodiments, the test sample and control sample are fluid samples.

In further embodiments, the level of expression of SLC9A3R1 RNA and the level of expression of at least one of enolase I RNA, cytokeratine 18 RNA, triosephosphate isomerase RNA, SFN RNA, and/or HPRT RNA are detected in the test cell sample and the control sample. In particular embodiments, the level of expression of SLC9A3R1 RNA and the level of expression of at least one of enolase I RNA, cytokeratine 18 RNA, triosephosphate isomerase RNA, SFN RNA, and/or HPRT RNA are detected by contacting the test sample and the control sample with a nucleic acid binding agent selected from the group consisting of RNA, cDNA, cRNA, and RNA-DNA hybrids. In more particular embodiments, the nucleic acid binding agent is immobilized on a solid support. In still more particular embodiments, the level of expression of SLC9A3R1, enolase I RNA, cytokeratine 18 RNA, triosephosphate isomerase RNA, SFN RNA, and/or HPRT RNA in the test sample is at least 1.5 times greater than the level of expression of SLC9A3R1 in the control sample.

In certain embodiments, the test sample is isolated from a tissue of a patient suffering from ovarian cancer, breast cancer, lung cancer, prostate cancer, and colon cancer. In other embodiments, the test sample is isolated from a patient suffering from non-small cell lung carcinoma.

### BRIEF DESCRIPTION OF THE FIGURES

The foregoing and other objects of the present invention, the various features thereof, as well as the invention itself may be more fully understood from the following description, when read together with the following accompanying drawings:

FIG. 1 is a graphic representation showing a scatter plot in which each dot represents the results of RNA expression analyses for SLC9A3R1 isolated from tissues in normal subjects (“Normal”) (N=61 samples) and tumor tissues from breast cancer patients (“Tumor”) (N=79 samples).

FIG. 2 is a graphic representation showing a scatter plot in which each dot represents the results of real-time PCR and microarray analyses of RNA expression levels that show the RNA expression levels of SLC9A3R1 in normal tissues (“Normal”) (N=56 samples) and tumor samples from breast cancer patients (“Tumor”) (N=67 samples). SLC9A3R1 expression levels are measured by micro-array and Real-time PCR in normal (control) and tumor (test samples).

FIG. 3 is a graphic representation showing a scatter plot in which each dot represents the RNA expression levels of SLC9AR1 in normal breast tissues (“Normal”) (N=25 samples) and tumor samples from breast cancer patients (“Tumor”) (N=37 samples) by Real-time PCR on formalin-fixed and paraffin-embedded tissue placed on microscope slides.

FIG. 4 is a graphic representation showing a scatter plot in which each dot represents the microarray experiment establishing the RNA expression levels of SLC9A3R1 in normal ovarian tissues (“Normal”) (N=77 samples) and tumor samples from ovarian cancer patients (“Tumor”) (N=62 samples). SLC9A3R1 expression levels are determined from micro-array experiments using normal (control) and tumor (test samples).

FIG. 5 is a graphic representation showing a scatter plot in which each dot represents the microarray experiment establishing the RNA expression levels of SLC9A3R1 in normal lung tissues (“Normal”) (N=15 samples) and tumor samples from lung (Non-small cell lung cancer or NSCLC) cancer patients (“Tumor”) (N=11 samples).

FIG. 6 is a graphic representation showing a scatter plot in which each dot represents the real-time PCR experiments determining the level of SLC9A3R1 RNA expression in normal lung tissues (“Normal”) (N=15 samples) and tumor samples from lung (Non-small cell lung cancer or NSCLC) cancer patients (“Tumor”) (N=11 samples).

FIG. 7 is a series of photographic representations of the results of Western blot experiments showing the relative levels of SLC9A3R1 protein expression in various normal breast tissues (“Normal”) (N=27 samples) and various breast cancer samples (“Tumor”) (N=72 samples).

FIG. 8 is a graphic representation showing a direct ELISA analysis of SLC9A3R1 protein expression in normal breast tissues (“Normal”) (N=27 samples) and breast cancer samples (“Tumor”) (N=72 samples).

FIG. 9 is a series of photographic representations showing Western blot analyses of relative levels of SLC9A3R1 protein expression in various normal ovarian tissues (“Normal”) (N=36 samples) and various tumor samples from ovarian cancer patients (“Tumor”) (N=34 samples).

FIG. 10 is a photographic representation of Western blot experiments showing the level of protein expression of SLC9A3R1 in blood/serum sample from a normal subject (“Normal”) and sample from an ovarian cancer patient (“Tumor”) as well as recombinant SLC9A3R1, which acted as a positive control.

FIG. 11 is a graphic representation of a optimization experiments to determine the appropriate conditions for SLC9A3R1 detection by Sandwich ELISA using different concentrations (0.01 μg/ml, 0.1 μg/ml, and 1.0 μg/ml) of a monoclonal antibody (“Capture Antibody”) and different concentrations (1000 ng/ml, 500 ng/ml, and 100 ng/ml) of a polyclonal antibody (“Detection Antibody).

FIG. 12 is a graphic representation of a standard curve showing the results of optimization experiments of SLC9A3R1 protein detection using a monoclonal capture antibody and polyclonal anti-SLC9A3R1 antibodies in an ELISA utilizing PBS or 3% BSA containing PBS.

FIG. 13 is a graphic representation showing the effects of BSA and increasing serum concentrations on SLC9A3R1 detection by Sandwich ELISA.

FIG. 14 is a graphic representation showing the level of SLC9A3R1 RNA expression, expressed as a ratio of expression of SLC9A3R1 in patient samples (BRT, LT, or OVT) and their respective normal RNA pool (BRN, LN, or OVN) as a reference, in non-small cell lung carcinoma (L) (T=11, N=15), breast carcinoma (Br) (T=79, N=61), and ovarian adenocarcinoma (OV) (T=62, N=77) patients compared to normal tissues. Results are expressed as a ratio of expression of SLC9A3R1 in patient samples and their respective normal.

FIG. 15 is a graphic representation of experiments showing the level of triosephosphate Isomerase (or TPI) RNA expression in non-small cell lung carcinoma (LT). (N=11), breast carcinoma (BrT) (N=79), and ovarian adenocarcinoma (OVT) (N=62) patients in comparison to lung normal tissues (LN) (N=15), breast normal tissues (BrN) (N=61), and ovarian normal tissues (OVN) (N=77). Results are expressed as a ratio of expression of triosephosphate isomerase in the patient samples and their respective normal RNA pool as reference.

FIG. 16 is a graphic representation of experiments showing the level of stratrifin (SFN)RNA expression of in non-small cell lung carcinoma (LT) (N=11), breast carcinoma (BrT) (N=79), and ovarian adenocarcinoma (OVT) (N=62) patients compared to lung normal tissues (LN) (N=15), breast normal tissues (BrN) (N=61), and ovarian normal tissues (OVN) (N=77). Results are expressed as a ratio of expression of Stratifin in the patient samples and their respective normal RNA pool as reference.

FIG. 17 is a graphic representation of experiments showing the level of cytokeratin 18 RNA expression of in non-small cell lung carcinoma (LT) (N=11), breast carcinoma (BrT) (N=79), and ovarian adenocarcinoma (OVT) (N=62) patients compared to lung normal tissues (LN) (N=15), breast normal tissues (BrN) (N=61), and ovarian normal tissues (OVN) (N=77). Results are expressed as a ratio of expression of Cytokeratin 18 in the patient samples and their respective normal RNA pool as reference.

FIG. 18 is a graphic representation of experiments showing the level of alpha enolase I RNA expression of in non-small cell lung carcinoma (LT) (N=11), breast carcinoma (BrT) (N=79), and ovarian adenocarcinoma (OVT) (N=62) patients compared to lung normal tissues (LN) (N=15), breast normal tissues (BrN) (N=61), and ovarian normal tissues (OVN) (N=77). Results are expressed as a ratio of expression of alpha enolase I in the patient samples and their respective normal RNA pool as reference.

FIG. 19 is a graphic representation of experiments showing the level of CRABP II RNA expression in non-small cell lung carcinoma (LT) (N=11), breast carcinoma (BrT) (N=79), and ovarian adenocarcinoma (OVT) (N=62) patients in comparison to lung normal tissues (LN) (N=15), breast normal tissues (BrN) (N=61), and ovarian normal tissues (OVN) (N=77). Results are expressed as a ratio of expression of CRAB-PII in patient samples and their respective normal RNA pool as reference.

FIG. 20 is a graphic representation of experiments showing the level of HPRT RNA expression of in non-small cell lung carcinoma (LT) (N=11), breast carcinoma (BrT) (N=79), and ovarian adenocarcinoma (OVT) (N=62) patients compared to lung normal tissues (LN) (N=15), breast normal tissues (BrN) (N=61), and ovarian normal tissues (OVN) (N=77). Results are expressed as normalized ratio of HPRT between the patient samples and the H23 tumor lung cell line calibrator.

FIG. 21 is a graphic representation showing the levels of RNA expression of Alpha enolase I, triosephosphate isomerase, Stratafin (or SNF), cytokeratin 18, SLC9A3R1, and CRAPBII in non-small cell lung carcinoma patients compared to normal lung tissues, expressed as a ratio of biomarker expression in the patient samples and their respective normal RNA pool as a reference. Results are expressed as a ratio of expression of the biomarkers in the patient samples and their respective normal RNA pool as reference.

FIG. 22 is a graphic representation showing the levels of RNA expression of Alpha enolase I, triosephosphate isomerase, Stratafin (or SNF), cytokeratin 18, SLC9A3R1, and CRAPBII in the breast carcinoma patients compared to normal breast tissues, expressed as a ratio of biomarker expression in the patient samples and their respective normal RNA pool as a reference. Results are expressed as a ratio of expression of the biomarkers in the patient samples and their respective normal RNA pool as reference.

FIG. 23 is a graphic representation showing the levels of RNA expression of Alpha enolase I, triosephosphate isomerase, Stratafin (or SNF), cytokeratin 18, SLC9A3R1, and CRAPBII in the ovarian carcinoma patients compared to normal ovarian tissues, expressed as a ratio of biomarker expression in the patient samples and their respective normal RNA pool as a reference. Results are expressed as a ratio of expression of the biomarkers in the patient samples and their respective normal RNA pool as reference.

### DETAILED DESCRIPTION OF THE INVENTION

Patent and scientific literature referred to herein establishes knowledge that is available to those of skill in the art. The issued US patents, allowed applications, published foreign applications, and references, including GenBank database sequences, that are cited herein are hereby incorporated by reference to the same extent as if each was specifically and individually indicated to be incorporated by reference.

### 1.1. General

The present invention provides, in part, methods and kits for diagnosing, detecting, or screening a test sample, such as a fluid sample, for tumorigenic potential and neoplastic characteristics such as aberrant growth. The invention also allows for the improved clinical management of tumors by providing a method that detects the expression level of a gene or genes identified as markers for cancer.

Typically, a gene will affect the phenotype of the cell through its expression at the protein level. Mutations in the coding sequence of the gene can alter its protein product in such a way that the protein does not perform its intended function appropriately. Some mutations, however, affect the levels of protein expressed in the cell without altering the functionality of the protein, itself. Such mutations directly affect the phenotype of a cell by changing the delicate balance of protein expression in a cell. Therefore, an alteration in a gene's overall activity can be measured by determining the level of expression of the protein product of the gene in a cell.

Accordingly, one aspect of the invention provides a method for diagnosing cancer in a cell. The method utilizes protein-targeting agents to identify proteins, such as SLC9A3R1, in a potentially cancerous cell sample or potentially cancerous serum or fluid sample. Increased levels of expression of particular protein markers in a cell or serum or fluid sample and a decreased expression level of other protein markers in a cell or serum or fluid sample indicate the presence of a neoplasm.

As used herein, “about” means a numeric value having a range of ±10% around the cited value. For example, a range of “about 1.5 times to about 2 times” includes the range “1.35 times to 2.2 times” as well as the range “1.65 times to 1.8 times,” and all ranges in between.

As used herein, the term “greater than” means more than, such as when the level of expression for a particular marker in test sample is detectably more than the level of expression for the same marker in a control sample. In these circumstances, expression analyses are qualitatively determined. The level of expression for a marker can also be determined quantitatively in test and control samples. In quantitative studies, the level of expression for a marker in a test sample is greater than the level of expression for the same marker in a control sample when the level of expression in the test sample is quantifiably determined to be at least about 10% more than the level of expression in the control sample.

As used herein, the term “protein-targeting agent” means a molecule capable of binding or interacting with a protein or a portion of a protein. Such binding or interactions can include ionic bonds, van der Waals interactions, London forces, covalent bonds, and hydrogen bonds. The target protein can be bound in a receptor-binding pocket, on its surface, or any other portion of the protein that is accessible to binding or interactions with a molecule. Protein-targeting agents include, but are not limited to, proteins, peptides, ligands, peptidomimetic compounds, inhibitors, organic molecules, aptamers, or combinations thereof.

As used herein, the term “inhibitor” means a compound that prevents a biomolecule, e.g., a protein, nucleic acid, or ribozyme, from completing or initiating a reaction. An inhibitor can inhibit a reaction by competitive, uncompetitive, or non-competitive means. Exemplary inhibitors include, but are not limited to, nucleic acids, proteins, small molecules, chemicals, peptides, peptidomimetic compounds, and analogs that mimic the binding site of an enzyme. In some embodiments, the inhibitor can be nucleic acid molecules including, but not limited to, siRNA that reduce the amount of functional protein in a cell.

As used herein, the term “tumorigenic potential” means ability to give rise to either benign or malignant tumors. Tumorigenic potential may occur through genetic mechanisms such as mutation or through infection with vectors such as viruses and bacteria.

The term “cancer” refers herein to a disease condition in which a tissue or cells exhibit aberrant, uncontrolled growth and/or lack of contact inhibition. A cancer can be a single cell or a tumor composed of hyperplastic cells. In addition, cancers can be malignant and metastatic, spreading from an original tumor site to other tissues in the body. In contrast, some cancers are localized to a single tissue of the body.

As used herein, a “cancer cell” is a cell that shows aberrant cell growth, such as increased, uncontrolled cell proliferation and/or lack of contact inhibition. A cancer cell can be a hyperplastic cell, a cell from a cell line that shows a lack of contact inhibition when grown in vitro, or a cancer cell that is capable of metastasis in vivo. In addition, cancer cells include cells isolated from a tumor or tumors. As used herein, a “tumor” is a collection of cells that exhibit the characteristics of cancer cells. Non-limiting examples of cancer cells include melanoma, ovarian cancer, ovarian cancer, renal cancer, osteosarcoma, lung cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, and thymoma. Cancer cells are also located in the blood at other sites, and include, but are not limited to, lymphoma cells, melanoma cells, sarcoma cells, leukemia cells, retinoblastoma cells, hepatoma cells, renal cancer cells, osteosarcoma cells, myeloma cells, glioma cells, mesothelioma cells, and carcinoma cells.

Cancer cells may also have the ability to metastasize to other tissues in the body. Metastasis is the process by which a cancer cell is no longer confined to the tumor mass, and enters the blood stream, where it is transported to a second site. Upon entering the other tissue, the cancer cell gives rise to a second situs for the disease and can take on different characteristics from the original tumor. Nevertheless, the new tumor retains characteristics from the tissue from which it derives, allowing for clinical identification of the type of cancer no matter where in the body a cancer cell or group of cells metastasizes. The process of metastasis has been studied extensively and is known in the art (see, e.g., Hendrix et al. (2000) Breast Cancer Res. 2(6): 417-22).

In certain embodiments of the invention, the cancer cell sample is obtained from a metastasized tumor or group of cells. The metastasized cells may be isolated from tissues including, but not limited to, blood, bone marrow, lymph node, liver, thymus, kidney, brain, skin, gastrointestinal tract, breast, and prostate.

The term “protein markers” as used herein means any protein, peptide, polypeptides, group of peptides, polypeptides or proteins expressed from a gene, whether chromosomal, extrachromosomal, endogenous, or exogenous, which may produce a cancerous or non-cancerous phenotype in the cell or the organism.

As used herein, “gene” means any deoxyribonucleic acid sequence capable of being translated into a protein or peptide sequence. The gene is a DNA sequence that may be transcribed into an mRNA and then translated into a peptide or protein sequence. Extrachromosomal sources of nucleic acid sequences can include double-strand DNA viral genomes, single-stranded DNA viral genomes, double-stranded RNA viral genomes, single-stranded RNA viral genomes, bacterial DNA, mitochondrial genomic DNA, cDNA or any other foreign source of nucleic acid that is capable of generating a gene product.

Protein markers can have any structure or conformation, and can be in any location within a cell, including on the cell surface. Protein markers can also be secreted from the cell into an extracellular matrix or directly into the blood or other biological fluid. Protein markers can be a single polypeptide chain or peptide fragments of a polypeptide. Moreover, they can also be combinations of nucleic acids and polypeptides as in the case of a ribosome. Protein markers can have any secondary structure combination, any tertiary structure, and come in quaternary structures as well.

One useful protein marker used to identify a neoplastic disease is SLC9A3R1 protein. Examples of SLC9A3R1 amino acid sequences include, but are not limited to, GenBank Accession Nos. AAH49220, NP_{—}001075814, NP_{—}067605, NP_{—}036160, NP_{—}004243, CAM21605, AAI02808, EAW89191, EAW89190, NP_{—}001071320, AAH85141, AAH53350, AAH11777, AAH03361, AAH01443, and EAW89189. Other useful protein markers include CRAB-PII (GenBank Accession Nos. P22935, P51673, P30370, AAA80225.1, P29373, and Q5pXY7), enolase I (GenBank Accession Nos. P06733, Q53FT9, Q9BT62, and Q96GV1), cytokeratin 18 (GenBank Accession Nos. NP_{—}954657, CAA31375, and NP_{—}000215), triosephosphate isomerase (GenBank Accession Nos. AAB59511, AAH70129, EAW88721, EAW88722, EAW88723, AAB51316, and NP_{—}000356), SFN (GenBank Accession Nos. NP_{—}006133, NP_{—}061224, CAB92118, CAM14836, AAH23552, AAH02995, AAH00995, and AAH00329), and HPRT (GenBank Accession Nos. AAB21292, AAB21291, AAB21289, AAB25009, Q64531, 1Z7G_A, 1Z7G_B, 1Z7G_C, 1Z7G_D, and AAA96232.).

As used herein, the term “test fluid sample” is a fluid that is obtained or isolated from a subject potentially suffering from a neoplastic disease. A fluid sample is isolated from urine, blood, lymph, pleural fluid, pus, marrow, cartilaginous fluid, saliva, seminal fluid, menstrual blood, and spinal fluid. Fluid samples can be isolated from tissues isolated from a subject. For instance, the tissues can be isolated from organs or tissues including, but not limited to, brain, kidney, blood, cartilage, lung, ovary, lymph nodes, salivary glands, breast, prostate, testes, uterus, skin, bone, and bone marrow. Fluid samples potentially include a neoplastic cell or group of cells. A test fluid sample can also be obtained from necrotic material isolated from a tumor or tumors. Such cell or group of cells may show aberrant cell growth, such as increased, uncontrolled cell proliferation and/or lack of contact inhibition. The test fluid sample can include, for example, a cancer cell that can be a hyperplastic cell, a cell from a cell line that shows a lack of contact inhibition when grown in vitro, or a cancer cell that is capable of metastasis in vivo.

As used herein, the term “test cell sample” refers to a cell, group of cells, or cells isolated from potentially cancerous tumor tissues. A test cell sample is one that potentially exhibits tumorigenic potential, metastatic potential, or aberrant growth in vivo or in vitro. A test cell sample can be isolated from tissues including, but not limited to, blood, bone marrow, spleen, lymph node, liver, lung, colon, thymus, kidney, brain, skin, gastrointestinal tract, eye, breast, and prostate.

As used herein, the term “non-neoplastic control cell sample” refers to a cell or group of cells that is exhibiting noncancerous normal characteristics for the particular cell type from which the cell or group of cells was isolated. A control cell sample does not exhibit tumorigenic potential, metastatic potential, or aberrant growth in vivo or in vitro. A control cell sample can be isolated from normal tissues in a subject that is not suffering from cancer. It may not be necessary to isolate a control cell sample each time a cell sample is tested for cancer as long as the nucleic acids isolated from the normal control cell sample allow for probing against the focused microarray during the testing procedure.

In another aspect, the invention provides methods for diagnosing cancer in a test cell sample by detecting SLC9A3R1 protein using a dipstick assay, Western blots, dot blots, and Enzyme-Linked Immunosorbent Assays (“ELISA's”).

SLC9A3R1 can also be detected with different cancer markers using a protein microarray. The methods can be practiced using a microarray composed of capture probes affixed to a derivatized solid support such as, but not limited to, glass, nylon, metal alloy, or silicon. Non-limiting examples of derivatizing substances include aldehydes, gelatin-based substrates, epoxies, poly-lysine, amines and silanes. Techniques for applying these substances to solid surfaces are well known in the art. In useful embodiments, the solid support can be comprised of nylon.

For purposes of the invention, the term “capture probe” is intended to mean any agent capable of binding a gene product in a complex cell sample or fluid sample. Capture probes can be disposed on the derivatized solid support utilizing methods practiced by those of ordinary skill in the art through a process called “printing” (see, e.g., Schena et. al., (1995) Science, 270(5235): 467-470). The term “printing”, as used herein, refers to the placement of spots onto the solid support in such close proximity as to allow a maximum number of spots to be disposed onto a solid support. The printing process can be carried out by, e.g., a robotic printer. The VersArray CHIP Writer Prosystem (BioRad Laboratories) using Stealth Micro Spotting Pins (Telechem International, Inc, Sunnyvale, Calif.) is a non-limiting example of a chip-printing device that can be used to produce a focused microarray for this aspect. The capture probes may be antibodies, fragments thereof, or any other molecules capable of binding a protein (herein termed “protein capture probes”). These probes may be attached to a solid support at predetermined positions.

The level of expression of SLC9A3R1 in the potentially cancerous test cell sample or potentially cancerous test fluid sample is compared to the level of expression of SLC9A3R1 in a non-neoplastic control cell or control fluid sample of the same tissue type. If the expression of SLC9A3R1 in the potentially cancerous cell or fluid sample is greater than the expression of SLC9A3R1 in the non-neoplastic control cell or fluid sample, then cancer is indicated. In some embodiments, the test cell or fluid sample is tumorigenic if the level of expression of SLC9A3R1 in the potentially cancerous cell or fluid sample is 1.5 times greater than the level of expression of SLC9A3R1 in the non-neoplastic control cell or fluid sample. In some embodiments, the test cell or fluid sample is tumorigenic if the level of expression of SLC9A3R1 in the potentially cancerous cell or fluid sample is at least 1.5 times greater than the level of expression of SLC9A3R1 in the non-neoplastic control cell or fluid sample. The test cell or fluid sample may be tumorigenic if the level of expression of SLC9A3R1 in the potentially cancerous cell or fluid sample is at least 2 times greater, at least 4 times greater, at least 6 times greater, between 8 and 12 times greater, at least 15 times greater, or at least 20 times greater than the level of expression of SLC9A3R1 in the non-neoplastic control cell or non-neoplastic fluid sample.

In embodiments in which test tissue and cell samples are used, cell samples can be isolated from human tumor tissues using means that are known in the art (see, e.g., Vara et al. (2005) Biomaterials 26(18):3987-93; Tyer et al. (1998) J. Biol. Chem. 273(5):2692-7). For example, the cell sample can be isolated from the ovary of a human patient with ovarian cancer. Other cancer cells that can be obtained include, but are not limited to, prostate cancer cells, melanoma cancer cells, osteosarcoma cancer cells, glioma cells, colon cancer cells, lung cancer cells, breast cancer cells, and leukemia cells. Cancer cells can metastasize to distant locations in the body. Non-limiting sites of metastases can include, but are not limited to, ovarian, bone, blood, lung, skin, brain, adipose tissue, muscle, gastrointestinal tissues, hepatic tissues, and kidney. Alternatively, the cell test or control cell sample can be obtained from a cell line. Cell lines can be obtained commercially from various sources (e.g., American Type Culture Collections, Mannassas, Va.). Alternatively, cell lines can be produced using techniques well known in the art.

In addition, the cell sample can be a cell line. Cancer cell lines can be created by one with skill in the art and are also available from common sources, such as the ATCC cell biology collections (American Type Culture Collections, Mannassas, Va.).

The present invention allows for the detection of cancer in tissues that are of mixed cellular populations such as a mixture of cancer cells and normal cells. In such cases, cancer cells can represent as little as 40% of the tissue isolated for the present invention to determine that the cell sample is tumorigenic. For example, the cell sample can be composed of 50% cancer cells for the present invention to detect tumorigenic potential. Cell samples composed of greater than 50% tumorigenic cells can also be used in the present invention. It should be noted that cell samples can be isolated from tissues that are less than 40% tumorigenic cells as long as the cell sample contains a portion of cells that are at least 40% tumorigenic.

In the present invention, levels of expression of housekeeping proteins are used to normalize the signal obtained between patients. As used herein, the term “housekeeping proteins” refers to any protein that has relatively stable or steady expression at the protein level during the life of a cell. Housekeeping proteins can be protein markers that show little difference in expression between cancer cells and normal cells in a particular tissue type. Examples of housekeeping proteins are well known in the art, and include, but are not limited to, isocitrate lyase, acyltransferase, creatine kinase, TATA-binding protein, hypoxanthine phosphoribosyl transferase 1, and guanine nucleotide binding protein, beta polypeptide 2-like 1 (see, e.g., Pandey et al. (2004) Bioinformatics 20(17): 2904-2910). In addition, the housekeeping proteins are used to identify the proper signal level by which to compare the cell sample signals between proteins from different or independent experiments.

Another aspect of the invention provides a method of diagnosing cancer in a fluid sample. In this method, expression of SLC9A3R1 in the fluid sample is measured. Expression levels for SLC9A3R1 can be determined using any techniques known in the art. Useful ways to determine such expression levels include, but not limited to, Western blot, protein microarrays, dipstick assays, dot blots, and Enzyme-Linked Immunosorbent Assays (“ELISA”) (see, e.g., U.S. Pat. Nos. 6,955,896; 6,087,012; 3,791,932; 3,850,752; and 4,034,074). Such examples are not intended to limit the potential means for determining the expression of a protein marker in a cell sample. Expression levels of markers in or by potentially cancerous cell samples and normal control cell samples can be compared using standard statistical techniques known to those of skill in the art (see, e.g., Ma et al., (2002) Methods Mol. Biol. 196:139-45).

The fluid sample can be isolated from a human patient by a physician and tested for expression of SLC9A3R1 using a dipstick or any other method that relies on a solid support, solid state binding, change in color, or electric current. In addition, the cancer cell sample can be isolated from an organism that develops a tumor or cancer cells including, but not limited to, mouse, rat, horse, pig, guinea pig, or chinchilla. Cell samples can be stored for extended periods prior to testing or tested immediately upon isolation of the cell sample from the subject. Cell samples can be isolated by non-limiting methods such as surgical excision, aspiration from soft tissues such as adipose tissue or lymphatic tissue, biopsy, or removed from the blood. These methods are known to those of skill in the art.

In certain embodiments, the level of expression of anti-SLC9A3R1 antibodies in a fluid sample is detected. The level of expression of anti-SLC9A3R1 antibodies in a cell sample is detected using ELISA, western blot, and dot blot. The level of expression of anti-SLC9A3R1 antibodies can be detected using antibodies or fragments thereof, which are directed against anti-USIDOCS SLC9A3R1 antibodies. The level of expression of anti-SLC9A3R1 antibodies can be detected using antibody fragments (e.g., Fab, F(ab)₂, and Fv) or whole antibodies.

A normal or ovarian cancer cell sample can be isolated from a human patient by a physician and tested for expression of protein markers using a dipstick or any other method that relies on a solid support, solid state binding, change in color, or electric current. In addition, the cancer cell sample can be isolated from an organism that develops a tumor or cancer cells including, but not limited to mammals such as mouse, rat, horse, pig, guinea pig, or chinchilla. Cell samples can be isolated by non-limiting methods such as surgical excision, aspiration from soft tissues such as adipose tissue or lymphatic tissue, biopsy, or removed from the blood. These methods are known to those of skill in the art. Cell samples can be stored for extended periods prior to testing or tested immediately upon isolation of the cell sample from the subject.

### 1.2. Nucleic Acid Binding Agents

In another aspect, the method of detecting cancer includes detecting a level of expression of SLC9A3R1 RNA in a test fluid sample (i.e., neoplastic test fluid sample) and comparing the level of expression of SLC9A3R1 RNA detected in the test fluid sample to the level of expression of SLC9A3R1 RNA detected in the non-neoplastic control fluid sample. If the level of expression of SLC9A3R1 RNA is greater in the test fluid sample than in the non-neoplastic control fluid sample, then cancer is indicated.

In still another aspect, the method of detecting cancer includes detecting a level of expression of SLC9A3R1 RNA in a test cell sample (i.e., neoplastic test fluid sample) and comparing the level of expression of SLC9A3R1 RNA detected in the test cell sample to the level of expression of SLC9A3R1 RNA detected in the non-neoplastic control cell sample. If the level of expression of SLC9A3R1 RNA is greater in the test cell sample than in the non-neoplastic control cell sample, then cancer is indicated.

As used herein, “nucleic acid binding agent” means a nucleic acid capable of hybridizing with a particular target nucleic acid sequence. Nucleic acid binding agents include any structure that can hybridize with a target nucleic acid such as an mRNA. Nucleic acids can include, but are not limited to, DNA, RNA, RNA-DNA hybrids, siRNA, and aptamers. Moreover, any detectable labels can be used so long as the label does not affect the hybridizing of the nucleic acid with its targeting. Labels include, but are not limited to, fluorophores, chemical dyes, radiolabels, chemiluminescent compounds, colorimetric enzymatic reactions, chemiluminescent enzymatic reactions, magnetic compounds, and paramagnetic compounds.

Examples of SLC9A3R1 nucleic acid sequences detected in the present invention include, but are not limited to, GenBank Accession Nos. NM_{—}004252, NM_{—}012030, NM_{—}021594, BC102807, BC085141, AK128474, BC001443, BC049220, BC053350, BC011777, and BC003361. Other useful nucleic acid markers include CRAB-PII (GenBank Accession Nos. AR035503.1, AR035502.1, AR035501.1, U23407, M68867, L01528, AH001884, M87539, and M87538), enolase I (GenBank Accession Nos. X16288.1, AK222517.1, BX537400.1, X84907.1, and M14328.1), cytokeratine 18 (GenBank Accession Nos. NG_{—}008351, NM_{—}000224, and NM_{—}199187), triosephosphate isomerase (GenBank Accession Nos. M10036.1, X69723.1, BC007086.1, and AK222638.1), SFN (GenBank Accession Nos. NM_{—}006142, NM_{—}139323, NM_{—}018754, and NM_{—}006826), and HPRT (GenBank Accession Nos. NG_{—}003031).

In certain embodiments, a focused microarray can be used to detect the levels of expression of SLC9A3R1 with other markers. The term “focused microarray” as used herein refers to a device that includes a solid support with capture probe(s) affixed to the surface of the solid support. In some embodiments, the focused microarray has nucleic acids attached to a solid support. Typically, the support consists of silicon, glass, nylon or metal alloy. Solid supports used for microarray production can be obtained commercially from, for example, Genetix Inc. (Boston, Mass.). Moreover, the support can be derivatized with a compound to improve nucleic acid association. Exemplary compounds that can be used to derivatize the support include aldehydes, poly-lysine, epoxy, silane containing compounds and amines. Derivatized slides can be obtained commercially from Telechem International (Sunnyvale, Calif.).

In the case of nucleic acid binding agents, nucleic acid sequences that are selected for detecting SLC9A3R1 expression may correspond to regions of low homology between genes, thereby limiting cross-hybridization to other sequences. Typically, this means that the sequences show a base-to-base identity of less than or equal to 30% with other known sequences within the organism being studied. Sequence identity determinations can be performed using the BLAST research program located at the NIH website (world wide web at ncbi.nlm.nih.gov/BLAST). Alternatively, the Needleman-Wunsch global alignment algorithm can be used to determine base homology between sequences (see Cheung et al., (2004) FEMS Immunol. Med. Micorbiol. 40(1): 1-9.). In addition, the Smith-Waterman local alignment can be used to determine a 30% or less homology between sequences (see Goddard et al., (2003) J. Vector Ecol. 28:184-9). Expression levels for the SLC9A3R1 can be determined using techniques known in the art, such as, but not limited to, immunoblotting, quantitative RT-PCR, microarrays, RNA blotting, and two-dimensional gel-electrophoresis (see, e.g., Rehman et al. (2004) Hum. Pathol. 35(11):1385-91; Yang et al. (2004) Mol. Biol. Rep. 31(4):241-8). Such examples are not intended to limit the potential means for determining the expression of a gene marker in a breast cancer fluid sample.

Other useful nucleic acid binding agents are specific for CRAB-PII, enolase I, cytokeratine 18, triosephosphate isomerase, SFN, and HPRT. These agents can be used in combination with SLC9A3R1 to detect neoplastic disease. In particular embodiments, a plurality of CRAB-PII, enolase I, cytokeratine 18, triosephosphate isomerase, SFN, and HPRT are detected with SLC9A3R1 in a neoplastic test fluid or cell sample. In such embodiments, the level of expression of at least one of CRAB-PII, enolase I, cytokeratine 18, triosephosphate isomerase, SFN, and HPRT is 1.5 times greater in a test fluid or cell sample than the level of expression of the same markers in a control fluid or cell sample. In other embodiments, the level of expression of at least one of CRAB-PII, enolase I, cytokeratine 18, triosephosphate isomerase, SFN, and HPRT is 2 times greater in a test fluid or cell sample than the level of expression of the same markers in a control fluid or cell sample. In more embodiments, the level of expression of at least one of CRAB-PII, enolase I, cytokeratine 18, triosephosphate isomerase, SFN, and HPRT is 5 times greater in a test fluid or cell sample than the level of expression of the same markers in a control fluid or cell sample. In still more embodiments, the level of expression of at least one of CRAB-PII, enolase I, cytokeratine 18, triosephosphate isomerase, SFN, and HPRT is 10 or more times greater in a test fluid or cell sample than the level of expression of the same markers in a control fluid or cell sample. The nucleic acid sequences of CRAB-PII, enolase I, cytokeratine 18, triosephosphate isomerase, SFN, and HPRT are SEQ ID NOS: 2, 3, 4, 5, 6, and 7, respectively.

### 1.3. Protein-Targeting Agents

Protein marker expression is used to identify tumorigenic potential. Protein markers, such as SLC9A3R1, can be obtained by isolation from a cell sample, or a fluid sample, using any techniques available to one of ordinary skill in the art (see, e.g., Ausubel et. al., Current Protocols in Molecular Biology, Wiley and Sons, New York, N.Y., 1999). Isolation of protein markers, including SLC9A3R1, from the potentially tumorigenic cell sample, or from a fluid sample obtained from a patient potentially suffering or suffering from neoplastic disease, allows for the generation of target molecules, providing a means for determining the expression level of the protein markers in the potentially tumorigenic cell or fluid sample as described below. The protein markers, such as SLC9A3R1, can be isolated from a tissue or fluid sample isolated from a human subject. The SLC9A3R1 and other protein markers can be isolated from a cytoplasmic fraction or a membrane fraction of the sample. Protein isolation techniques known in the art include, but are not limited to, column chromatography, spin column chromatography, and protein precipitation. SLC9A3R1 can be isolated using methods that are taught in, for example, Ausubel et al., Current Protocols in Molecular Biology, Vol. 1, John Wiley & Sons, Inc., (1993).

The invention provides protein-targeting agents such as binding agents, e.g., antibodies or antigen binding fragments thereof. These embodiments are described in detail below. Other potential protein targeting agents include, but are not limited to, aptamers and ligands specific for SLC9A3R1 peptidomimetic compounds, peptides directed to the active sites of an enzyme, and nucleic acids.

Inhibitors can also be used as protein targeting agents to bind to protein markers. Useful inhibitors are compounds that bind to a target protein, and normally reduce the “effective activity” of the target protein in the cell or cell sample. Inhibitors include, but are not limited to, antibodies, antibody fragments such as “Fv,” “F(ab′)2,” “F(ab),” “Dab” and single chains representing the reactive portion of an antibody (“SC-Mab”), peptides, peptidomimetic compounds, and small molecules (see, e.g., Lopez-Alemany et al. (2003) Am. J. Hematol. 72(4): 234-42; Miles et al. (1991) Biochem. 30(6): 1682-91). Inhibitors can perform their functions through a variety of means including, but not limited to, non-competitive, uncompetitive, and competitive mechanisms. For instance, the triosephosphate isomerase 1 inhibitor N-hydroxy-4-phosphono-butanamide has been described previously (see, e.g., Verlinde et al (1989) Protein Sci. 1(12): 1578-84) and is useful.

Protein-targeting agents, including antibodies can also be conjugated to non-limiting materials such as magnetic compounds, paramagnetic compounds, proteins, nucleic acids, antibody fragments, or combinations thereof. Furthermore, antibodies can be disposed on an NPV membrane and placed into a dipstick. Antibodies can also be immobilized on a solid support at pre-determined positions such as in the case of a microarray. For instance, antibodies can be printed or cross-linked via their Fc regions to pre-derivatized surfaces of solid supports. In addition, antibodies can be cross-linked using bifunctional crosslinkers to a functionalized solid support. Such bifunctional crosslinking is well known in the art (see, e.g., U.S. Pat. Nos. 7,179,447; 7,183,373).

Crosslinking of proteins, such as antibodies, to a water-insoluble support matrix can be performed with bifunctional agents well known in the art including 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Bifunctional agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates can be employed for protein immobilization.

Protein-targeting agents can be detectably labeled. As used herein, “detectably labeled” means that a targeting agent is operably linked to a moiety that is detectable. By “operably linked” is meant that the moiety is attached to the protein-targeting agent by either a covalent or non-covalent (e.g., ionic) bond. Methods for creating covalent bonds are known (see, e.g., Wong, S. S., Chemistry of Protein Conjugation and Cross-Linking, CRC Press 1991; Burkhart et al., The Chemistry and Application of Amino Crosslinking Agents or Aminoplasts, John Wiley & Sons Inc., New York City, N.Y., 1999).

According to the invention, a “detectable label” is a moiety that can be sensed. Such labels can be, without limitation, fluorophores (e.g., fluorescein (FITC), phycoerythrin, rhodamine), chemical dyes, or compounds that are radioactive, chemiluminescent, magnetic, paramagnetic, promagnetic, or enzymes that yield a product that may be colored, chemiluminescent, or magnetic. The signal is detectable by any suitable means, including spectroscopic, photochemical, biochemical, immunochemical, electrical, optical or chemical means. In certain cases, the signal is detectable by two or more means. In certain embodiments, protein targeting agents include fluorescent dyes, radiolabels, and chemiluminescent labels, which are examples that are not intended to limit the scope of the invention (see, e.g., Gruber et al. (2000) Bioconjug. Chem. 11(5): 696-704).

For example, protein-targeting agents may be conjugated to Cy5/Cy3 fluorescent dyes. These dyes are frequently used in the art (see, e.g., Gruber et al. (2000) Bioconjug. Chem. 11(5): 696-704). The fluorescent labels can be selected from a variety of structural classes, including the non-limiting examples such as 1- and 2-aminonaphthalene, p,p′diaminostilbenes, pyrenes, quaternary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopridinium salts, hellebrigenin, tetracycline, sterophenol, benzimidazolyl phenylamine, 2-oxo-3-chromen, indole, xanthen, 7-hydroxycoumarin, phenoxazine, salicylate, strophanthidin, porphyrins, triarylmethanes, flavin, xanthene dyes (e.g., fluorescein and rhodamine dyes); cyanine dyes; 4,4-difluoro-4-bora-3a,4a-diaza-s-indacene dyes and fluorescent proteins (e.g., green fluorescent protein, phycobiliprotein).

### 1.4. Antibodies for Detection of SLC9A3R1

Aspects of the present invention utilize monoclonal and polyclonal antibodies as protein targeting agents directed specifically against certain cancer marker proteins, particularly SLC9A3R1. Other useful markers include CRAB-PII, enolase I, cytokeratine 18, triosephosphate isomerase, SFN, and HPRT. In certain embodiments, SLC9A3R1 is used alone as a protein marker to diagnose cancer. Anti-SLC9A3R1 protein antibodies, both monoclonal and polyclonal, for use in the invention are available from several commercial sources (e.g., Santa Cruz Biotechnology, Santa Cruz, Calif.; and Biogenesis, Inc., Kingston, N.H.). SLC9A3R1, CRAB-PII, enolase I, cytokeratine 18, triosephosphate isomerase, SFN, and HPRT antibodies can be administered to a patient orally, subcutaneously, intramuscularly, intravenously, or interperitoneally for in vivo detection and/or imaging.

As used herein, the term “polyclonal antibodies” means a population of antibodies that can bind to multiple epitopes on an antigenic molecule. A polyclonal antibody is specific to a particular epitope on an antigen, while the entire pool of polyclonal antibodies can recognize different epitopes. In addition, polyclonal antibodies developed against the same antigen can recognize the same epitope on an antigen, but with varying degrees of specificity. Polyclonal antibodies can be isolated from multiple organisms including, but not limited to, rabbit, goat, horse, mouse, rat, and primates. Polyclonal antibodies can also be purified from crude serums using techniques known in the art (see, e.g., Ausubel, et al., Current Protocols in Molecular Biology, Vol. 1, pp. 4.2.1-4.2.9, John Wiley & Sons, Inc., 1996).

The term “monoclonal antibody”, as used herein, refers to an antibody obtained from a population of substantially homogenous antibodies, i.e., the individual antibodies comprising the population are identical except for possible naturally occurring mutations that may be present in minor amounts. Monoclonal antibodies are highly specific, being directed against a single antigenic site. By their nature, monoclonal antibody preparations are directed to a single specific determinant on the target. Novel monoclonal antibodies or fragments thereof mean in principle all immunoglobulin classes such as IgM, IgG, IgD, IgE, IgA, or their subclasses or mixtures thereof. Non-limiting examples of subclasses include the IgG subclasses IgG1, IgG2, IgG3, IgG2a, IgG2b, IgG3, or IgGM. The IgG subtypes IgG1/κ and IgG2b/κ are also included within the scope of the present invention. Antibodies can be obtained commercially from, e.g., BioMol International LP (Plymouth Meeting, Pa.), BD Biosciences Pharmingen (San Diego, Calif.), and Cell Sciences, Inc. (Canton, Mass.).

The monoclonal antibodies herein include hybrid and recombinant antibodies produced by splicing a variable (including hypervariable) domain of an anti-SLC9A3R1 protein antibody with a constant domain (e.g., “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab)₂, and Fv), so long as they exhibit the desired biological activity. (See, e.g., U.S. Pat. No. 4,816,567; Mage and Lamoyi, in Monoclonal Antibody Production Techniques and Applications, (Marcel Dekker, Inc., New York 1987, pp. 79-97). Thus, the modified “monoclonal” indicates the character of the antibody as being obtained from a substantially homogeneous population of antibodies, and is not to be construed as requiring production of the antibody by any particular method. For example, the monoclonal antibodies to be used in accordance with the present invention can be made by the hybridoma method (see, e.g., Kohler and Milstein (1975) Nature 256:495) or can be made by recombinant DNA methods (U.S. Pat. No. 4,816,567). The monoclonal antibodies can also be isolated from phage libraries generated using the techniques described in the art (see, e.g., McCafferty et al (1990) Nature 348:552-554).

Alternative methods for producing antibodies can be used to obtain high affinity antibodies. Antibodies can be obtained from human sources such as serum. Additionally, monoclonal antibodies can be obtained from mouse-human heteromyeloma cell lines by techniques known in the art (see, e.g., Kozbor (1984) J. Immunol. 133, 3001; Boerner et al., (1991) J. Immunol. 147:86-95). Methods for the generation of human monoclonal antibodies using phage display, transgenic mouse technologies, and in vitro display technologies are known in the art and have been described previously (see, e.g., Osbourn et al. (2003) Drug Discov. Today 8: 845-51; Maynard and Georgiou (2000) Ann. Rev. Biomed. Eng. 2:339-76; U.S. Pat. Nos. 4,833,077; 5,811,524; 5,958,765; 6,413,771; and 6,537,809).

Aspects of the invention also utilize polyclonal antibodies for the detection of SLC9A3R1, CRAB-PII, enolase I, cytokeratine 18, triosephosphate isomerase, SFN, and HPRT. They can be prepared by known methods or commercially obtained.

In addition, aptamers can be protein targeting agents. The term “aptamer,” used herein interchangeably with the term “nucleic acid ligand,” means a nucleic acid that, through its ability to adopt a specific three-dimensional conformation, binds to and has an antagonizing (i.e., inhibitory) effect on a target. The target of the present invention is SLC9A3R1, and hence the term SLC9A3R1 aptamer or nucleic acid ligand is used. The aptamer can bind to the target by reacting with the target, by covalently attaching to the target, or by facilitating the reaction between the target and another molecule. Aptamers may be comprised of multiple ribonucleotide units, deoxyribonucleotide units, or a mixture of both types of nucleotide residues. Aptamers may further comprise one or more modified bases, sugars or phosphate backbone units as described above.

Aptamers can be made by any known method of producing oligomers or oligonucleotides. Many synthesis methods are known in the art. For example, 2′-O-allyl modified oligomers that contain residual purine ribonucleotides, and bearing a suitable 3′-terminus such as an inverted thymidine residue (Ortigao et al., (1992) Antisense Res. Devel. 2:129-146) or two phosphorothioate linkages at the 3′-terminus to prevent eventual degradation by 3′-exonucleases, can be synthesized by solid phase beta-cyanoethyl phosphoramidite chemistry (Sinha et al., Nucleic Acids Res., 12:4539-4557 (1984)) on any commercially available DNA/RNA synthesizer. Purification can be performed either by denaturing polyacrylamide gel electrophoresis or by a combination of ion-exchange HPLC (Sproat et al., (1995) Nucleosides and Nucleotides, 14:255-273) and reversed phase HPLC. For use in cells, synthesized oligomers are converted to their sodium salts by precipitation with sodium perchlorate in acetone. Traces of residual salts may then be removed using small disposable gel filtration columns that are commercially available. As a final step the authenticity of the isolated oligomers may be checked by matrix assisted laser desorption mass spectrometry (Pieles et al., (1993) Nucleic Acids Res., 21:3191-3196) and by nucleoside base composition analysis.

There are several techniques that can be adapted for refinement or strengthening of the nucleic acid ligands binding to a particular target molecule or the selection of additional aptamers. One technique has been termed Selective Evolution of Ligands by Exponential Enrichment (SELEX). Compositions and methods for generating aptamer antagonists of the invention by SELEX and related methods are known in the art and taught in, for example, U.S. Pat. Nos. 5,475,096 and 5,270,163. The SELEX process in general is further described in, e.g., U.S. Pat. Nos. 5,668,264, 5,696,249, 5,670,637, 5,674,685, 5,723,594, 5,756,291, 5,811,533, 5,817,785, 5,958,691, 6,011,020, 6,051,698, 6,147,204, 6,168,778, 6,207,816, 6,229,002, 6,426,335, and 6,582,918.

1.5. Detection of SLC9A3R1 from Biological Fluids

An aspect of the present invention includes an assay for the detection of SLC9A3R1 protein using a protein-targeting agent to bind to the SLC9A3R1 protein. The SLC9A3R1 protein typically is a peptide, polypeptide, protein, glycoprotein, or protiolipid. The protein-targeting agent can comprise antigens and antibodies thereto; haptens and antibodies thereto; and hormones, ligands, vitamins, metabolites and pharmacological agents, and their receptors and binding substances. The protein-targeting agent may be an immunologically-active polypeptide or protein or molecular weight between 1,000 Daltons and 10,000,000 Daltons, such as an antibody or antigenic polypeptide or protein, or a hapten of molecular weight between 100 Daltons and 1,500 Daltons. Protein-targeting agents can bind to SLC9A3R1 protein that are obtained from biological fluids. As used herein, the term “biological fluids” means aqueous or semi-aqueous liquids isolated from an organism in which biological macromolecules may be identified or isolated. Biological fluids may be disposed internally as in the case of blood serum, bile, or cerebrospinal fluid. Biological fluids can be excreted as in the non-limiting cases of urine, saliva, sweat, tears, mucosal secretions, lacrimal secretions, seminal fluid, sperm, and sebaceous secretions.

For detection of markers in biological fluids, detection devices can be used that are in the form of a “dipstick.” Such devices are known in the art, and have been applied to detecting SLC9A3R1 protein in serum and other biological fluids (see, e.g. U.S. Pat. No. 4,390,343). In some instances, a dipstick-type device can be comprised of analytical elements where protein-targeting agents, such as antibodies, inhibitors, organic molecules, peptidomimetic compounds, ligands, organic compounds, or combinations thereof, are incorporated into a gel. The gel can be comprised of non-limiting substances such as agarose, gelatin or PVP (see, e.g., U.S. Pat. No. 4,390,343). The gel can be contained within an analytical region for reaction with a protein marker.

The “dipstick” format (exemplified in U.S. Pat. Nos. 5,275,785, 5,504,013, 5,602,040, 5,622,871 and 5,656,503) typically consists of a strip of porous material having a biological fluid sample-receiving end, a reagent zone and a reaction zone. As used herein, the term “reagent zone” means the area within the dipstick in which the protein-targeting agent and the SLC9A3R1 protein in the biological sample come into contact. By the term “reaction zone”, is meant the area within the dipstick in which an immobilized binding agent captures the protein-targeting agent/protein marker complex. As used herein, the term “binding agent” refers to any molecule or group of molecules that can bind, interact, or associate with a protein-targeting agent/protein marker complex.

In certain embodiments, the biological fluid sample is wicked along the assay device starting at the sample-receiving end and moving into the reagent zone. The protein marker(s) to be detected binds to a protein-targeting agent incorporated into the reagent zone, such as a labeled protein-targeting agent, to form a complex. For example, a labeled antibody can be the protein-targeting agent, which complexes specifically with the protein marker. In other examples, the protein-targeting agent can be a receptor that binds to a protein marker in a receptor:ligand complex. In yet other examples, an inhibitor is used to bind to a protein marker, thereby forming a complex with the protein marker targeted by the particular inhibitor. In some examples, peptidomimetic compounds are used to bind to SLC9A3R1 protein to mimic the interaction of a protein marker with a normal peptide. In other examples, the protein-targeting agent can be an organic molecule capable of associating with the protein marker. In all cases, the protein-targeting agent has a label. The labeled protein-targeting agent-protein marker complex then migrates into the reaction zone, where the complex is captured by another specific binding partner firmly immobilized in the reaction zone. Retention of the labeled complex within the reaction zone thus results in a visible readout.

A number of different types of other useful assays that measure the presence of a protein market are well known in the art. One such assay is an immunoassay. Immunoassays may be homogeneous, i.e. performed in a single phase, or heterogeneous, where antigen or antibody is linked to an insoluble solid support upon which the assay is performed. Sandwich or competitive assays may be performed. The reaction steps may be performed simultaneously or sequentially. Threshold assays may be performed, where a predetermined amount of analyte is removed from the sample using a capture reagent before the assay is performed, and only analyte levels of above the specified concentration are detected. Assay formats include, but are not limited to, for example, assays performed in test tubes, wells or on immunochromatographic test strips, as well as dipstick, lateral flow or migratory format immunoassays.

A lateral flow test immunoassay device may be used in this aspect of the invention. In such devices, a membrane system forms a single fluid flow pathway along the test strip. The membrane system includes components that act as a solid support for immunoreactions. For example, porous or bibulous or absorbent materials can be placed on a strip such that they partially overlap, or a single material can be used, in order to conduct liquid along the strip. The membrane materials can be supported on a backing, such as a plastic backing. The test strip includes a glass fiber pad, a nitrocellulose strip and an absorbent cellulose paper strip supported on a plastic backing.

Antibodies that specifically bind with the target protein marker are immobilized on the solid support. The antibodies can be bound to the test strip by adsorption, ionic binding, van der Waals adsorption, electrostatic binding, or by covalent binding, by using a coupling agent, such as glutaraldehyde. For example, the antibodies can be applied to the conjugate pad and nitrocellulose strip using standard dispensing methods, such as a syringe pump, airbrush, ceramic piston pump or drop-on-demand dispenser. A volumetric ceramic piston pump dispenser can be used to stripe antibodies that bind the analyte of interest, including a labeled antibody conjugate, onto a glass fiber conjugate pad and a nitrocellulose strip.

The test strip can be treated, for example, with sugar to facilitate mobility along the test strip or with water-soluble non-immune animal proteins, such as albumins, including bovine (BSA), other animal proteins, water-soluble polyamino acids, or casein to block non-specific binding sites.

### 1.6. Cancer Diagnosis and Prediction Analysis

Cancer diagnoses can be performed by comparing the levels of expression of a protein marker, such as SLC9A3R1, or a set of protein markers including SLC9A3R1 in a potentially neoplastic cell sample to the levels of expression for a protein marker or a set of protein markers in a normal control cell sample of the same tissue type. Alternatively, the level of expression of a protein marker, such as SLC9A3R1, or a set of protein markers in a potentially cancerous cell sample is compared to a reference pool of protein markers that represents the level of expression for a protein marker or a set of protein markers in a normal control population (herein termed “training set”). The training set also includes the data for a population that has a known tumor or class of tumors. This data represents the average level of expression that has been determined for the neoplastic cells isolated from the tumor or class of tumors. It also has data related to the average level of expression for a protein marker or set of protein markers for normal cells of the same cell type within a population. In these embodiments, the algorithm compares newly generated expression data for a particular protein marker or set of protein markers from a cell sample isolated from a patient containing potentially neoplastic cells to the levels of expression for the same protein marker or set of protein markers in the training set. The algorithm determines whether a cell sample is neoplastic or normal by aligning the level of expression for a protein marker or set of protein markers with the appropriate group in the training set. In certain embodiments, software for performing the statistical manipulations described herein can be provided on a computer connected by data link to a data generating device, such as a microarray reader.

Class prediction algorithms can be utilized to differentiate between the levels of expression of markers in a cell sample and the levels of expression of markers in a normal cell sample (Vapnik, The Nature of Statistical Learning Theory, Springer Publishing, 1995). Exemplary, non-limiting algorithms include, but are not limited to, compound covariate predictor, diagonal linear discriminant analysis, nearest neighbor predictor, nearest centroid predictor, and support vector machine predictor (Simon et al., Design and Analysis of DNA Microarray Investigations An Artificial Intelligence Milestone., Springer Publishing, 2003). These statistical tests are well known in the art, and can be applied to ELISA or data generated using other protein expression determination techniques such as dot blotting, Western Blotting, and protein microarrays (see, e.g., U.S. Appln. No. 2005/0239079).

It should be recognized that statistical analysis of the levels of expression of protein markers in a cell sample to determine cancer state does not require a particular algorithm or set of particular algorithms. Any algorithm can be used in the present invention so long as it can discriminate between statistically significant and statistically insignificant differences in the levels of expression of protein markers in a cell sample as compared to the levels of expression of the same protein markers in a normal cell sample of the same tissue type. In this case, a test sample is considered cancerous or malignant if the expression of one or more protein marker is above a cut-off value established for one or all markers in normal or control samples.

In some embodiments, an increased level of expression in the potentially cancerous cell sample, or fluid sample, indicates that cancer cells exist in the cell sample. In such cancerous samples, protein markers showing increased levels of expression include, but are not limited to, SLC9A3R1, as well as CRAB-PII, enolase I, cytokeratine 18, triosephosphate isomerase, SFN, and HPRT. The algorithm makes the class prediction based upon the overall levels of expression found in the cell sample as compared to the levels of expression in the training set. It should be noted that, in some instances, SLC9A3R1 can be used to classify a ? (YES it Can) sample as either neoplastic or normal. Two or more protein markers, including SLC9A3R1, can also be used to properly classify a cell sample as neoplastic or normal. In particular, three protein markers, including SLC9A3R1, can be used for classification purposes. Four protein markers, including SLC9A3R1, can be used to identify neoplastic cells within a cell sample. Five protein markers, including SLC9A3R1, can be used to identify neoplastic cells in a cell sample. Furthermore, six or more protein markers, including SLC9A3R1, can be used to properly classify cell samples into either the neoplastic cell class or the non-neoplastic cell class.

The type of analysis detailed above compares the level of expression for the protein marker(s) in the cell sample to a training set containing reference pools of protein that are representative of a normal population and a neoplastic population. In certain embodiments, the training set can be obtained with kits that can be used to determine the level of expression of protein marker(s) in a patient cell sample. Alternatively, an investigator can generate new training sets using protein expression reference pools that can be obtained from commercial sources such as Asterand, Inc. (Detroit, Mich.). Comparisons between the training sets and the cell samples are performed using standard statistical techniques that are well known in the art, and include, but are not limited to, the ArrayStat 1.0 program (Imaging Research, Inc., Brock University, St. Catherine's, Ontario, Calif.). Statistically significant increased levels of expression in the cell sample of protein marker(s) indicate that the cell sample contains a cancer cell or cells with tumorigenic potential. Also, standard statistical techniques such as the Student T test are well known in the art, and can be used to determine statistically significant differences in the levels of expression for protein markers in a patient cell sample (see, e.g., Piedra et al. (1996) Ped. Infect. Dis. J. 15: 1). In particular, the Student T test is used to identify statistically significant changes in expression using protein microarray analysis or ELISA analysis (see, e.g., Piedra et al. (1996) Ped. Infect. Dis. J. 15:1).

### 1.7. Protein Microarray

Protein microarrays can be prepared by methods disclosed in, e.g., U.S. Pat. Nos. 6,087,102, 6,139,831, and 6,087,103. In addition, protein-targeting agents conjugated to the surface of the protein microarray can be bound by detectably labeled protein markers isolated from a cell sample or a fluid sample. This method of detection can be termed “direct labeling” because the protein marker, which is the target, is labeled. In other embodiments, protein markers can be bound by protein-targeting agents, and then subsequently bound by a detectably labeled antibody specific for the protein marker. These methods are termed “indirect labeling” because the detectable label is associated with a secondary antibody or other protein-targeting agent. An overview of protein microarray technology in general can be found in Mitchell, Nature Biotech. (2002), 20:225-229, the contents of which are incorporated herein by reference.

### 1.8. Kits

Aspects of the invention additionally provide kits for detecting neoplasms such as ovarian, lung, breast, colon and prostate cancers in a cell or a fluid sample. The kits include targeting agents for the detection of SLC9A3R1 and CRAB-PII, enolase I, cytokeratine 18, triosephosphate isomerase, SFN, and/or HPRT. In certain embodiments, kits include targeting agents for the detection of SLC9A3R1. A patient that potentially has a tumor or the potential to develop a tumor (“in need thereof”) can be tested for the presence of a tumor or tumor potential by determining the level of expression of targeting agents in a cell or fluid sample derived from the patient.

The kit comprises labeled binding agents capable of detecting at least one of SLC9A3R1, CRAB-PII, enolase I, cytokeratine 18, triosephosphate isomerase, SFN, and/or HPRTin a biological sample, as well as means for determining the amount of these protein markers in the sample, and means for comparing the amount of the protein markers in the potentially cancerous sample with a standard (e.g., normal non-neoplastic control cells). The binding agents can be packaged in a suitable container. The kit can further comprise instructions for using the compounds or agents to detect the protein markers, as well as other neoplasm-associated markers. Such a kit can comprise, e.g., one or more antibodies, or fragments thereof as binding agents, that bind specifically to at least a portion of a protein marker.

In particular, kits comprise labeled binding agents capable of binding to and detecting SLC9A3R1, as well as means determining the amount of SLC9A3R1 in the sample, and means for comparing the amount of the protein markers in the potentially cancerous sample with a standard (e.g., normal non-neoplastic control cells). Such a kit can comprise, e.g., one or more antibodies, or fragments thereof as binding agents, that bind specifically to at least a portion of a SLC9A3R1.

The kit can also contain a probe for detection of housekeeping protein expression. These probes advantageously allow health care professionals to obtain an additional data point to determine whether a specific or general cancer treatment is working so SLC9A3R1 levels can be used to monitor the success of cancer treatment. The probes can be any binding agents such as labeled antibodies, or fragments thereof, specific for the housekeeping proteins. Alternatively or additionally, the probes can be inhibitors, peptidomimetic compounds, peptides and/or small molecules.

Data related to the levels of expression of the selected protein markers in normal tissues and neoplasms can be supplied in a kit or individually in the form of a pamphlet, document, floppy disk, or computer CD. The data can represent patient pools developed for a particular population (e.g., Caucasian, Asian, etc.) and is tailored to a particular cancer type. Such data can be distributed to clinicians for testing patients for the presence of a neoplasm such as an ovarian cancer. A clinician obtains the levels of expression for a protein marker or set of protein markers in a particular patient. The clinician then compares the expression information obtained from the patient to the levels of expression for the same protein marker or set of protein markers that had been determined previously for both normal control and cancer patient pools. A finding that the level of expression for the protein marker or the set of protein markers is similar to the normal patient pool data indicates that the cell sample obtained from the patient is not neoplastic. A finding that the level of expression for the protein marker or the set of protein markers is similar to the cancer patient pool data indicates that the cell sample obtained from the patient is neoplastic.

### 1.9. Testing

The diagnostic methods according to the invention were tested for their ability to diagnose cancer in test cell samples isolated from human subjects suffering from ovarian cancer, lung cancer, prostate cancer, hepatic cancer, pancreatic cancer, breast cancer, leukemia, sarcoma, melanoma, renal cancer, colon cancer, and osteosarchma.

The expression levels of SLC9A3R1 RNA and SLC9A3R1 protein in combination with other cancer markers were analyzed for differential expression in ovarian, breast and lung samples by Western blotting and focused microarray. The testing and results are described in detail below in the Examples.

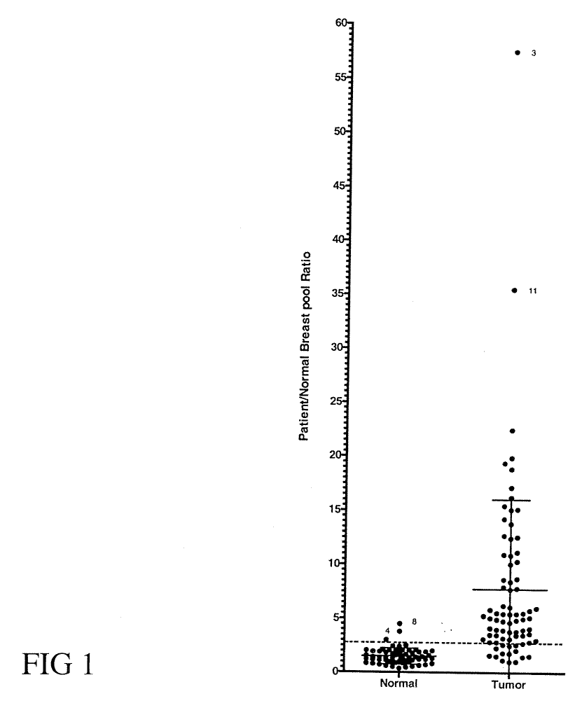

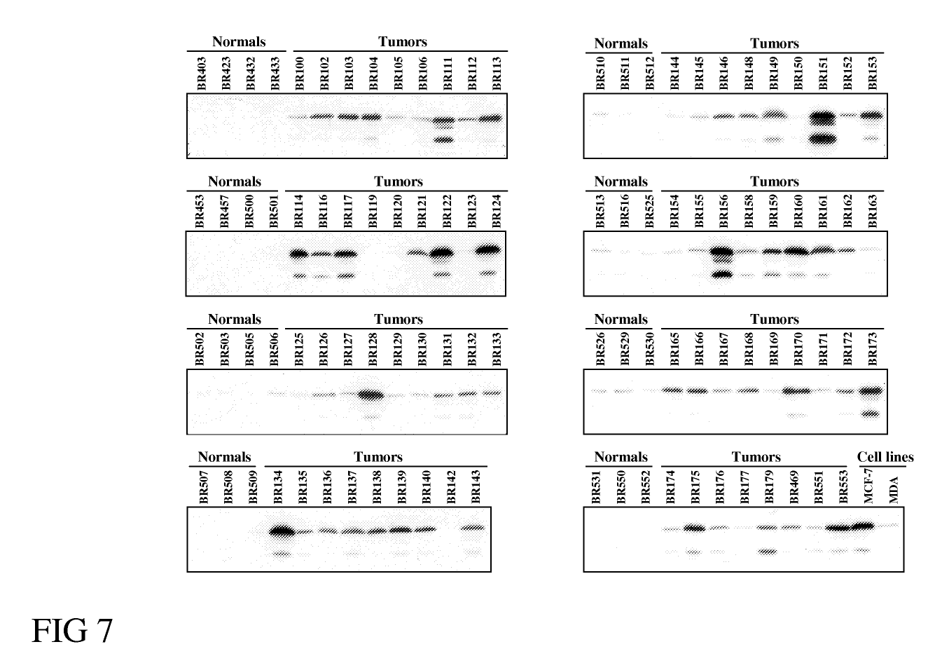

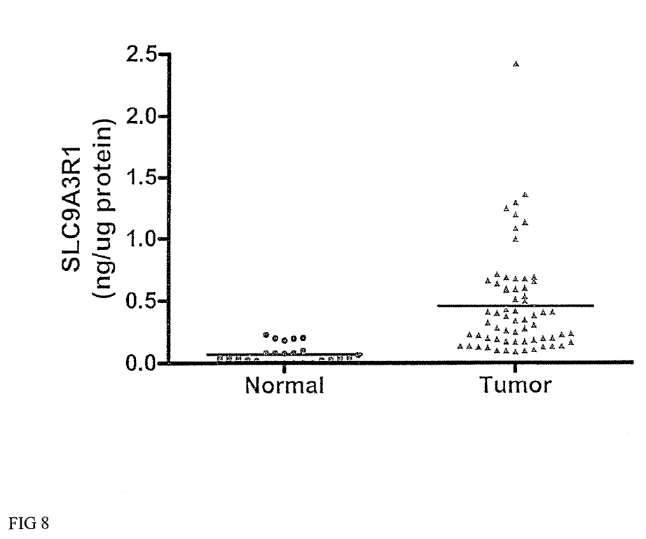

As shown in the Examples below, SLC9A3R1 RNA expression is increased in breast tumor tissues as compared to normal breast tissues (FIGS. 1-3). In addition, SLC9A3R1 protein expression is increased in tumor tissues as compared to normal breast tissues (FIGS. 7-8). These results indicate that the increase in SLC9A3R1 expression is a marker of the transformation of normal breast cells to neoplastic breast cells.

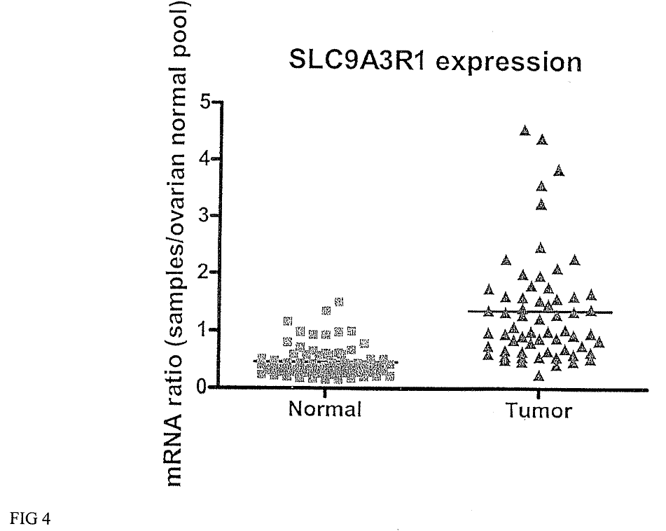

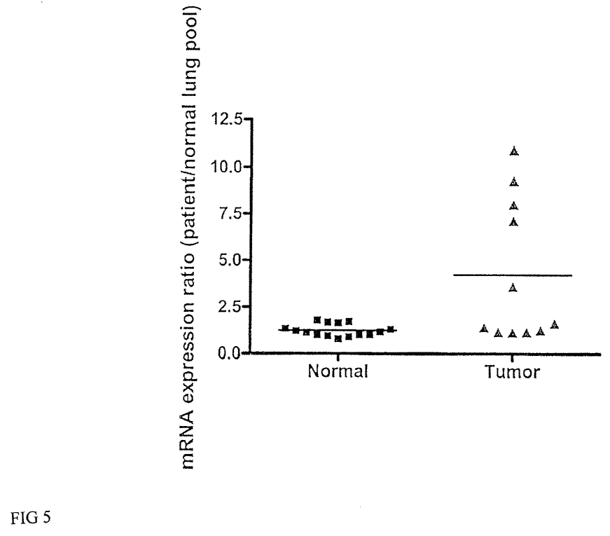

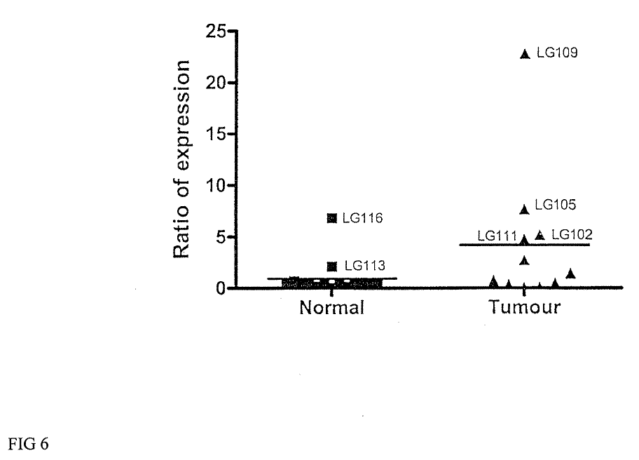

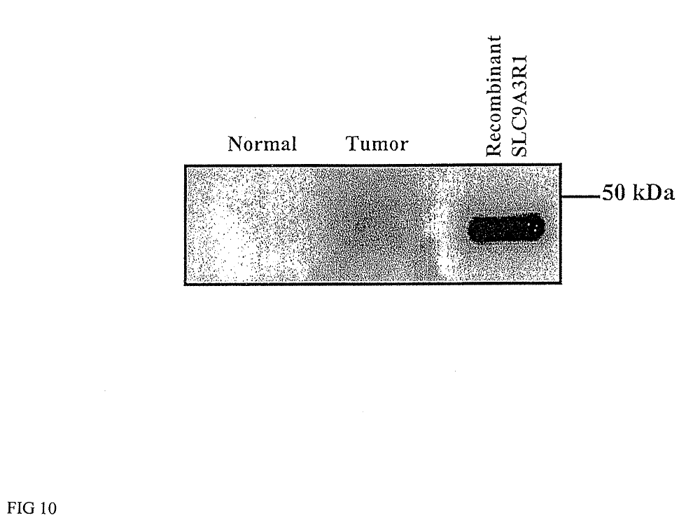

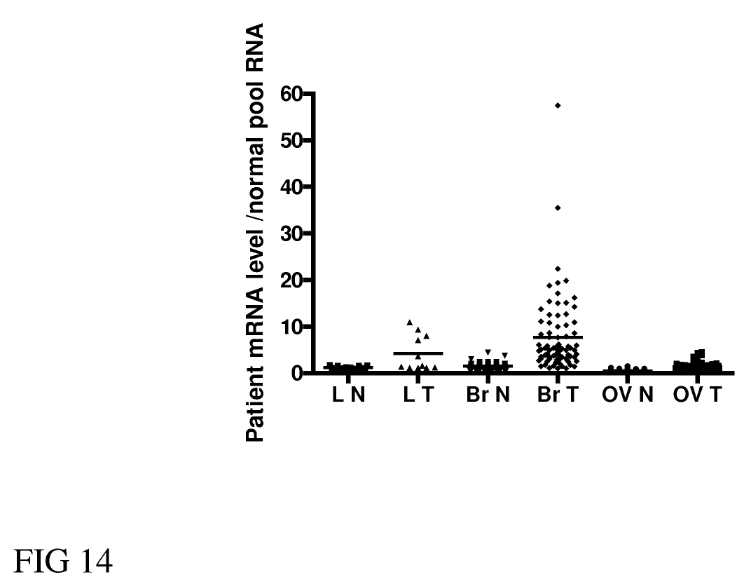

Increased expression of SLC9A3R1 RNA and protein was also observed in ovarian cancer patient samples as compared to normal tissue samples (FIGS. 4, 9 and 10). In addition, lung cancer samples showed higher levels of RNA expression as compared to normal lung tissues (FIGS. 5 and 6). As for breast, SLC9A3R1 overexpression is a marker of neoplastic disease in lung and ovarian tissues. FIG. 14 summarizes the results of the RNA experiments by showing a scatter plot of the expression levels found in breast, lung, and ovarian cancer patients and normal tissue-matched subjects.

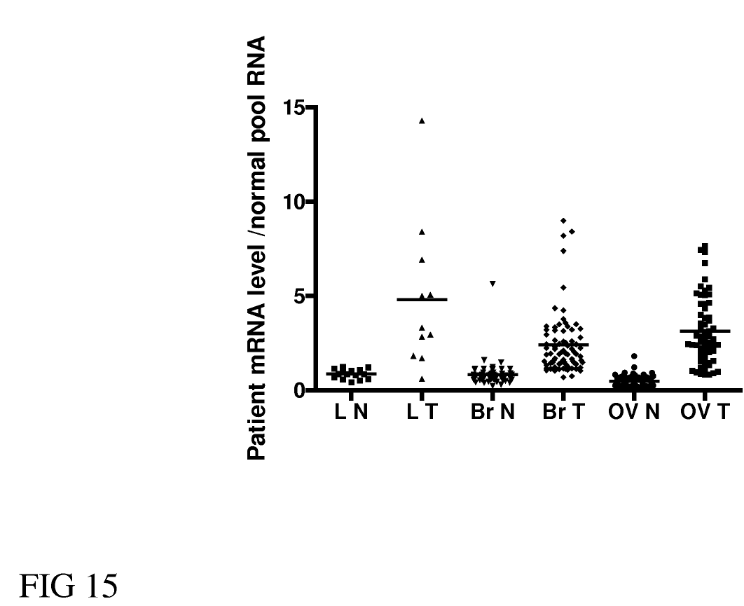

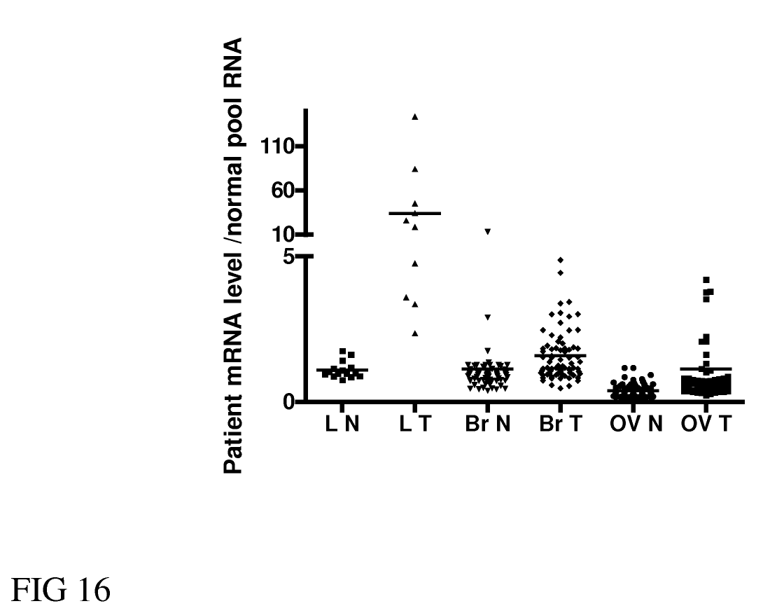

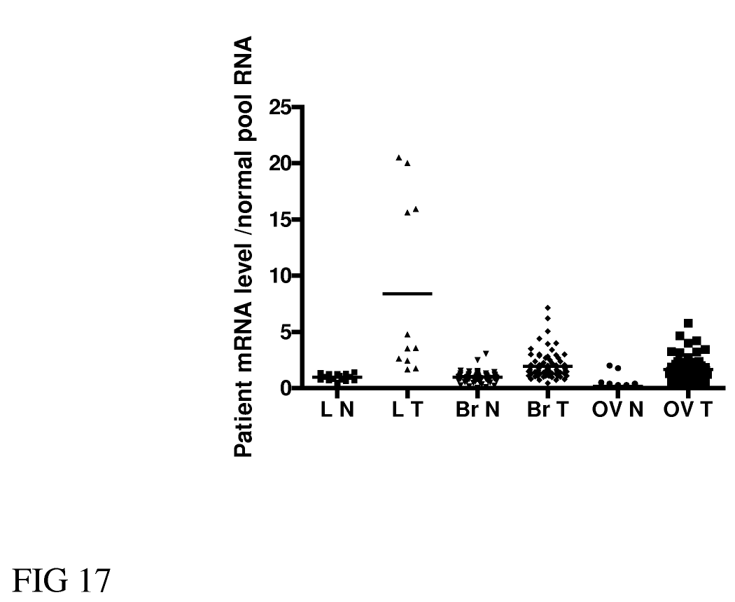

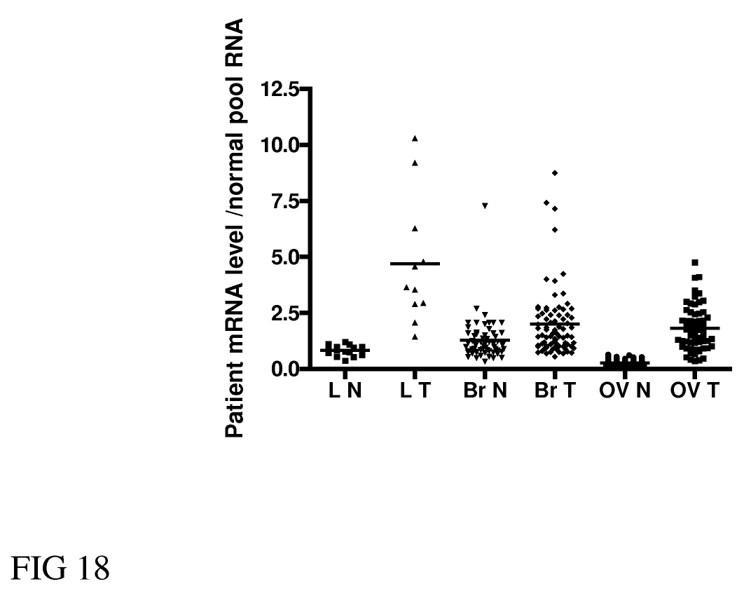

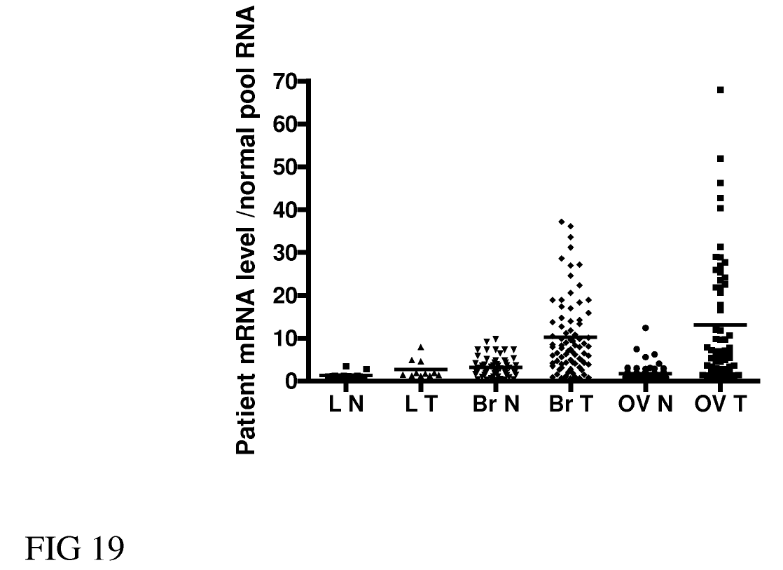

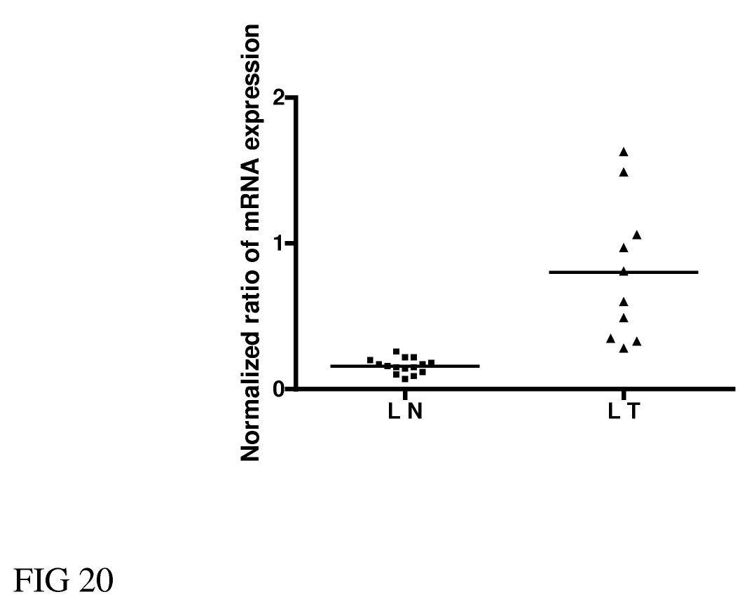

Furthermore, other markers were tested for differential expression in breast, ovarian, and lung tissues. As shown in FIGS. 15-19, triosephosphate isomerase, stratifin, cytokeratin 18, enolase (i.e., α-enolase), and CRAB-PII all showed increased RNA expression to varying degrees in breast, ovarian, and lung tumor tissues as compared to tissue matched controls. A sixth marker, HPRT, was increased in its levels of RNA expression in lung tumor tissues as compared to tissue-matched controls (FIG. 20). These results indicate that these proteins can be used as markers of neoplastic disease in combination with SLC9A3R1.

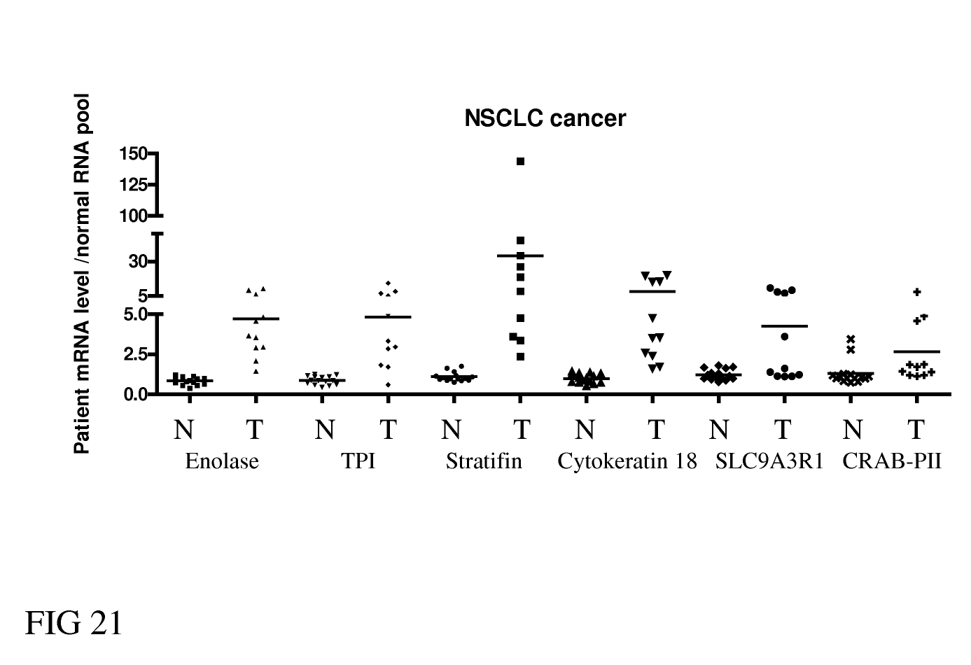

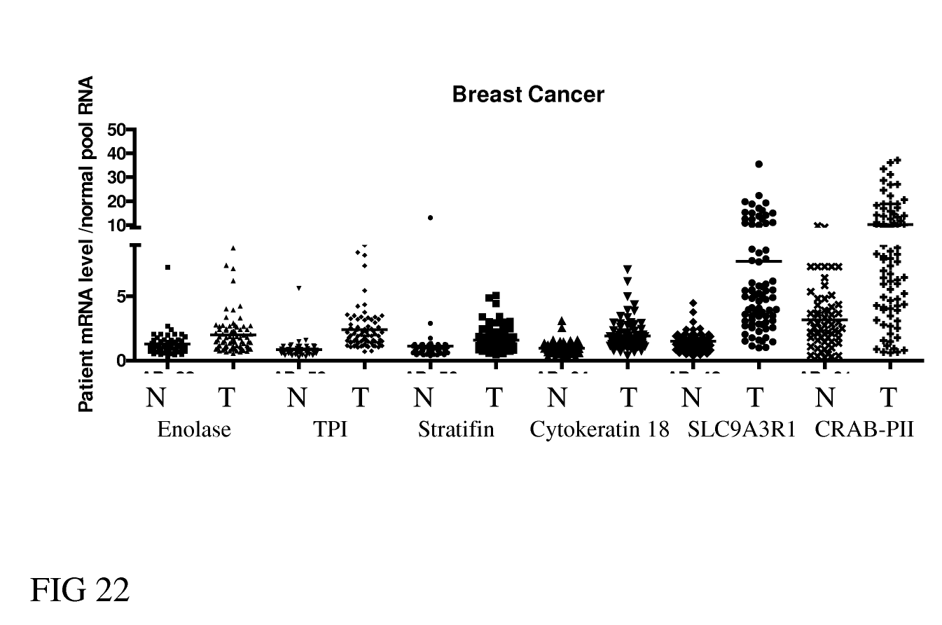

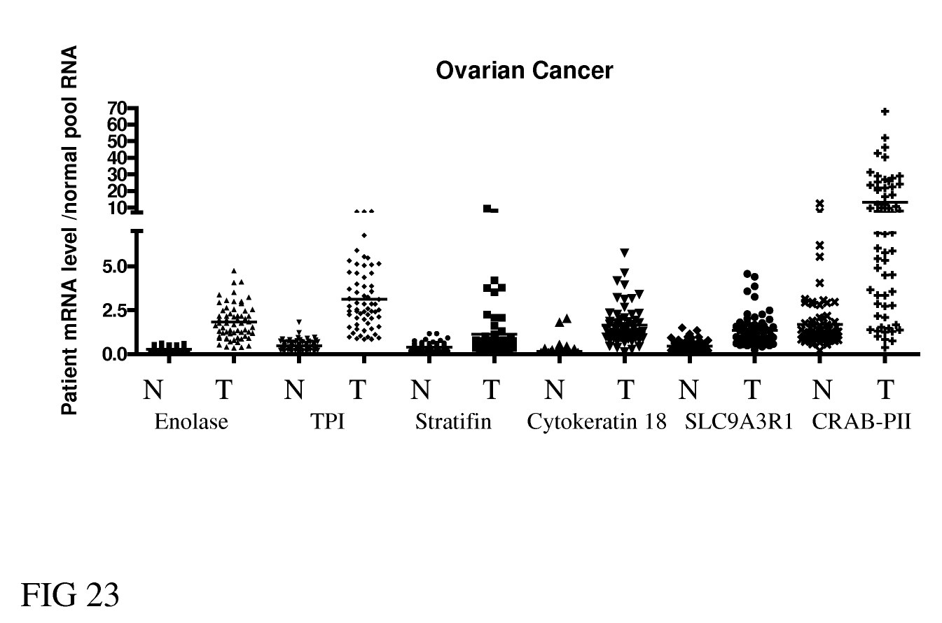

FIG. 21-23 show a compilation of the RNA expression results found in lung cancer tissues as compared to tissue-matched controls (FIG. 21), breast cancer tissues as compared to tissue-matched controls (FIG. 22), ovarian cancer tissues as compared to tissue-matched controls (FIG. 23). In all, these results, in combination with the results described in the Examples, indicate that SLC9A3R1 alone, or in combination with the other markers described herein, is a marker of neoplastic disease.

### EXAMPLES

Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Preparation and use of Focused Microarray for the Detection of SLC9A3R1 in Samples Obtained From Normal Ovarian Subjects and Ovarian Cancer Patients

1. Total RNA Isolation and cDNA Labeling

Patient tissues samples were obtained from Asterand, Inc. (Detroit, Mich.), Clinomics Biosciences, Inc (Watervliet, N.Y.) and Biochain Institute, Inc. (Hayward, Calif.). Each patient included in the study was screened against the same normal total RNA pool in order to compare them together. The tumor pool composed of 77 cases. The ovarian normal pool was composed of 62 cases.

For ovarian cell samples, human tissues were homogenized using a Polytron PT10-35 (Brinkmann, Mississauga, Canada) for 30 seconds at speed setting of 4 in the presence of 300 μl of 10 mM HEPES-Tris, pH 7.4, 150 mM NaCl, 1% Triton X-100, 1% sodium deoxycholic acid, 0.1% SDS, 1 mM EDTA and a cocktail of protease inhibitors from Roche Corp. (Laval, Qc, Canada). Cell lysis, in the case of cell and tissue samples, and RNA extraction was done with the RNEasy kit, (# 74104) (Qiagen, Inc., Valencia, Calif.) following the manufacturer's protocol. RNA was quantified by spectrophotometry using an Ultrospec 2000 spectrophotometer (Amersham-Biosciences, Corp., Piscataway, N.J.). RNA samples were dissolved in 10 mM Tris, pH 7.5 to determine the A_{260/280} ratios. Samples with ratios between 1.9 and 2.3 were kept for probe preparation, while samples with ratios lower than 1.9 were discarded. RNA samples were dissolved in 1 μl DEPC-H₂O for total nucleic acid quantification. Total RNA from control and treated samples was dried by speed vacuum using a Heto Vacuum centrifuge system (KNF Neuberger, Inc., Trenton, N.J.) at varying time intervals. The total RNA was resuspended in 10 μl of DEPC-H₂O and stored at −20° C. until the labeling reaction.

First strand cDNA labeling was accomplished using 1-15 μg total RNA (depending on the cell lines to be tested) for the resistant and the sensitive cell lines separately. Total RNA was incubated with 4 ng control positive Arabidopsis thaliana RNA, 3 μg of Oligo (dT)₁₂₋₁₈ primer (# Y01212) (Invitrogen, Corp., Carlsbad, Calif.), 1 μg PdN6 random primer (Amersham, #272166-01) for 10 min. at 65° C., and immediately put on ice for 1 min. The mixture was then diluted in 5× First strand buffer (250 mM Tris-HCl, pH 8.3; 375 mM KCl; 15 mM MgCl₂) containing 0.1 M DTT, 0.5 μM dNTPs mix (dTTP, dGTP, dATP) (Invitrogen, #10297-018), 0.05 μM dCTP (Invitrogen, #10297-018), 5 μM Cy3-dCTP (#NEL 576) (NEN Life Science/Perkin Elmer, Boston, Mass.), 2.5 μM Cy5-dCTP (#NEL 577) (NEN Life Science/Perkin Elmer, Boston, Mass.) and 400 units SuperScript III RNAse H⁻ RT (Invitrogen, #I 8064-014). After incubating the reaction mixture for 5 min. at 25° C., the reaction mixture was incubated at 42° C. for 90 min. Finally, a total of 400 units of SuperScript II RNAse H⁻ RT (Invitrogen, #18064-014) were added and the reaction was incubated at 42° C. for another 90 min.

Digestion of the labeled cDNA with 5 units RNAse H (#M0297S) (NEB, Beverly, Mass.) and 40 units RNAse A (Amersham, # 70194Y) was done at 37° C. for 30 min. The labeling probe was purified with the QIAquick PCR purification kit (Qiagen, Inc.) protocol with some modifications. Briefly, the reaction volume was completed to 50 μl with DEPC-H₂O and 2.7 μl of 12 M NaOAc pH 5.2 was added. The reaction was diluted with 200 μl PB buffer, put on the purification column, spun 15 sec. at 10 000 g, followed by 3 washes of 500 μl PE buffer (15 sec.; 10 000 g) and eluted 2 times in 50 μl DEPC-H₂O total (1 min.; 10 000 g). Frequency of incorporation and amount of cDNA labeled produced were evaluated for both labeled dCTPs by spectrophotometer (Ultrospec 2000, Pharmacia Biotech) at A₂₆₀ nm, A₅₅₀ nm, and A₆₅₀ nm. The labeling material was dried by speed vacuum (Heto Vacuum centrifuge system, LaboPort) and resuspended in 3.75 μl H₂O total for both Cy5 (resistant cell line) and Cy3 reactions (sensitive cell line).

### 2. Capture Probe Preparation

Capture probes, approximately 68 nucleotides in length, corresponding to targets of interest were designed using sequences showing less identity base to base (<30%) with other coding sequences (cds) submitted to NCBI bank. The comparisons between sequences were done by BLAST research (www.ncbi.nlm.nih.gov/BLAST). For BioChip ver1.0 and ver2.0, a basic melting point temperature at a salt concentration of 50 mM Na⁺ (Tm) for each capture probe was calculated: the overall average was 76.97° C.+/−3.72° C. GC nucleotide content averaged 51.2%+/−9.4%. For the present invention, two negative controls (68 bp of the antisense cds of the BRCP and nucleophosmin targets) were synthesized.

The SLC9A3R1 nucleic acid capture probe targets SLC9A3R1 GenBank Accession No. NM_{—}004252 (SEQ ID NO: 1). CRAB-PII nucleic acid capture probe targets GenBank Accession No. NM_{—}001878 (SEQ ID NO: 2). Enolase-1 nucleic acid capture probe targets GenBank Accession No. NM_{—}001428 (SEQ ID NO: 3). Cytokeratin 18 nucleic acid capture probe targets GenBank Accession No. NM_{—}000224 (SEQ ID NO: 4). Triosephosphate isomerase nucleic acid capture probe targets GenBank Accession No. U47924 (SEQ ID NO: 5). Stratifin nucleic acid capture probe targets GenBank Accession No. NM_{—}006142 (SEQ ID NO: 6). HPRT nucleic acid capture probe targets GenBank Accession No. NM_{—}000194 (SEQ ID NO: 7). Cytokeratin 18 nucleic acid capture probe targets GenBank Accession No. NM_{—}199187 (SEQ ID NO: 8).

The capture probe was synthesized by the BRI Institute (Biotechnology Research Institute, Clear Water Bay, Kowloon, Hong Kong, China) with the Expedilite™ Synthesizer at a coupling efficiency of over 99.5% (Applied Biosystems, Foster City, Calif.). The oligonucleotides were verified by polyacrylamide gel electrophoresis. Oligonucleotide quantification was done by spectrophotometry at A_{260 nm}.

### 3. Printing of Capture Probes and Production of the Focused Microarray

Prior to printing of capture probes, different dilutions of Arabidopsis thaliana chlorophyll synthetase G4 DNA (undiluted solutions at 0.15 μg/μl and at 0.2 μg/μl; 1:2; 1:4; 1:8; 1:16) were printed on each grid as a positive control, and for normalization of results. Preparation of Arabidopsis thaliana control capture probes was performed as follows. Briefly, five micrograms of a Midi preparation using a HiSpeed™ Plasmid Midi kit (Qiagen, Inc.) of the Arabidopsis thaliana plasmid was digested with 40 units of Sac I enzyme (NEB) for 2 hr. at 37° C., purified with the QIAquick PCR purification kit (Qiagen,) and verified by 1% agarose migration. In vitro transcription of 2 μg Sac I digestion was performed in 10× transcription buffer (400 mM Tris-HCl, pH 8.0; 60 mM MgCl₂; 100 mM DTT; 20 mM Spermidin) containing 2 μl of 10 mM NTP mix (Invitrogen), 20 units RNAse OUT (Invitrogen, #10777-019) and 50 units T7 RNA polymerase (NEB) for approximately 2 hr. to 30 hr. at 37° C. The reaction was then treated with 2 units DNAse I (Invitrogen) in 10× DNAse buffer (200 mM Tris-HCl pH 8.4; 20 mM MgCl₂; 500 mM KCl) for 15 min. at 37° C. The RNA was cleaned with the RNEasy kit (Qiagen) and quantified by spectrophotometry using an Ultrospec 2000 (Amersham Biosciences, Corp. Piscataway, N.J.).

After the control capture probes were generated and printed, the capture probes complementary to marker genes from the cancer cell samples were printed at concentrations of 25 μM in 50% DMSO on CMT-GAPS II Slides (# 40003) (Corning, 45 Nagog Park, Acton, Mass.) by the VersArray CHIP Writer Prosystems (BioRad Laboratories) with the Stealth Micro Spotting Pins (#SMP3) (Telechem International, Inc., Sunnyvale, Calif.). Each capture probe was printed in triplicate on duplicate grids. Buffer and Salmon Testis DNA (Sigma D-7656) were also printed for the BioChip analysis step. After printing was completed, the slides were dried overnight by incubation in the CHIP Writer chamber. Chips were then treated by UV (Stratagene, UV Stratalinker) at 600 mJoules and baked in an oven for 6-8 hr.

### 4. Quality Control of Focused Microarray

Prior to testing the invention on cancer cell samples, the focused microarray was tested at the BRI Institute (Kowloon Bay, Hong Kong). One slide for each printed batch was quality control tested using a terminal deoxynucleotidyl transferase (Tdt)-mediated nick end labeling assay protocol (see, e.g., Yeo et. al., (2004) Clin. Cancer Res. 10(24): 8687-96). Additionally, controls were performed to verify the specificity of the hybridization using three independent grids on the same focused microarray.

As a first quality control, a test was done by the BRI Institute on one slide for each batch printed with the following Tdt transferase protocol. Briefly, the slide was prehybridized in a Hybridization Chamber (#2551) (Corning, Inc., Life Sciences, 45 Nagog Park, Acton, Mass.) with 80 μl of preheated prehybridization buffer (5×SSC (750 mM NaCl; 75 mM sodium citrate); 0.1% SDS; 1% BSA (Sigma, #A-7888) at 37° C. for 30 min. Slides were washed in 0.1×SSC (15 mM NaCl; 1.5 mM sodium citrate) and air-dried. 50 μl of TdT reaction mixture [5×TdT buffer (125 mM Tris-HCl, pH 6.6, 1 M sodium cacodylate, 1.25 mg/ml BSA); 5 mM CoCl₂; 1 mM Cy3-dCTP (NEN Life Science, NEL 576); 50 units TdT enzyme (#27-0730-01) (Amersham BioSciences)], was added to the entire area of the BioChip. The slide was incubated in the Hybridization Chamber for 60 min. at 37° C., following by a first wash in 1×SSC (150 mM NaCl; 15 mM sodium citrate)/0.2% SDS (preheated at 37° C.) for 10 min., a second wash of 5 min. in 0.1×SCC (15 mM NaCl; 1.5 mM sodium citrate)/0.2% SDS at RT and finally a last wash of 5 min. at RT in 0.1×SSC (15 mM NaCl; 1.5 mM sodium citrate). The slide was scanned with the ScanArray™ Lite MicroArray Scanner (Packard BioSciences, Perkin Elmer, San Jose, Calif.).

As a second quality control step, the PARAGON™ DNA Microarray Quality Control Stain kit (Molecular Probes) was incubated with the microarray according to the manufacturer's recommendations.

5. Focused Microarray Hybridization with Labeled cDNA Probes

Focused microarray slides were pre-washed before the prehybridization step as follows. First, slides were washed for 20 min. at 42° C. in 2×SSC (300 mM NaCl; 30 mM sodium citrate)/0.2% SDS under agitation. The second wash was for 5 min. at RT in 0.2×SSC (30 mM NaCl, 3 mM Sodium citrate) under agitation, and then followed by a wash for 5 min. at RT in DEPC-H₂O with agitation. The slides were spin dried at 1000 g for 5 min. and prehybridized in Dig Easy Hyb Buffer (#1,603,558) (Roche Diagnostics Corp., Indianapolis, Ind.) containing 400 μg Bovine Serum Albumin (Roche, #711,454) at 42° C. in humid chamber for 3 hr. then washed 2 times in DEPC-H₂O, and once in Isopropanol (Sigma, 1-9516) and spun dry at 1000 g for 5 min.

To the mixed Cy5/Cy3 probe, 15 μg Baker tRNA (#109,495) (Roche Diagnostics Corp., Indianapolis, Ind.) and 1 μg Cot-1DNA (Roche, #1,581,074) were added and the probe was incubated 5 min. at 95° C., put on ice for 1 min., and diluted with 14 μl Dig Easy Hyb buffer (Roche, #1,603,558). After a 2 min. spin at 100 g, the probe was incubated at 42° C. for at least 5 min.

The three supergrids on the slide were separated by a Jet-Set Quick Dry TOP Coat 101 line (#FX268) (L'Oreal, Paris, FR). Each probe was added to its respective supergrid and covered by a preheated (42° C.) coverslip (Mandel, #S-104 84906). The slide was incubated at 42° C. in humid chamber for at least 15 hr.

The coverslips were removed by dipping in 1×SSC (150 mM NaCl; 15 mM sodium citrate)/0.2% SDS solution preheated at 50° C.). The slide was washed three times for 5 min. with agitation in 1×SSC (150 mM NaCl; 15 mM sodium citrate)/0.2% SDS solution preheated at 50° C.), and then washed three times with agitation in 0.1×SSC (15 mM NaCl; 1.5 mM sodium citrate)/0.2% SDS solution preheated at 37° C.). Finally, the slide was washed once in 0.1×SSC (15 mM NaCl; 1.5 mM sodium citrate) with agitation for 5 min. The slide was dipped several times in DEPC-H₂O and spun dry at 1000 g for 5 min.

### 6. Scanning and Statistical Analysis

The slides were scanned with a ScanArray™ Lite MicroArray Scanner (Packard BioSciences, Perkin Elmer, San Jose, Calif.) and the analysis was performed with a QuantArray® Microarray Analysis software version 3.0 (Packard BioSciences, Perkin Elmer, San Jose, Calif.).

The QuantArray® data results were analyzed according to the following procedures. All analysis of the results was performed with the spot background subtracted values for Cy5 and Cy3. Spots with lower signal ratio to noise lower than 1.5 were discarded. Normalization of the ratios with the spike positive control (Arabidopsis thaliana) was done to have a ratio equal to one for that control on each slide. Slides were discarded on which the negative and/or positive controls did not work. Also, slides were discarded with high background and with different mean no offset correction (ArrayStat software). Mean for each target was calculated with at least six different experiments (including two reciprocal labeling reactions), each experiment using different total RNA preparations. Statistical analysis was accomplished with the ArrayStat 1.0 (Imaging Research Inc., Brock University, St. Catherine's, Ontario, Calif.). A log transformation of the ratio data is followed by a Student T test for two independent conditions using a proportional model without offsets at a p<0.05 threshold. Significant increases (ratio Cy5/Cy3 higher than 1.5) or decreases (ratio Cy5/Cy3 lower than 0.5) were considered to be significant if the p value was lower than 0.05.

### 7. Results.

Increased levels of SLC9A3R1 mRNA were detected in tumor samples obtained patients suffering from ovarian cancer compared to normal subjects (FIG. 4). Tumor samples from patients suffering from ovarian cancer averaged about 3 times higher levels of SLC9A3R1 mRNA expression than found in normal subjects (FIG. 4).

### Example 2

### Preparation and Use of Focused Microarray to Detect SLC9A3R1 in Samples Obtained from Normal Breast Subjects and Breast Cancer Patients

1. Total RNA Isolation and cDNA Labeling

Patient tissues samples were obtained from Asterand, Inc. (Detroit, Mich.), Clinomics Biosciences, Inc (Watervliet, N.Y.) and Biochain Institute, Inc. (Hayward, Calif.). Each patient included in the study was screened against the same normal total RNA pool in order to compare them together. The tumor pool was composed of 79 cases. The breast normal pool was composed of 61 cases.

Patient samples, capture probes, and microarrays were prepared as described in Example 1. Microarray experiments were performed as described in Example 1. Levels of RNA expression are determined as ratio or folds of expression of SLC9A3R1 in each control (or normal) and tumor (test) sample relative to SLC9A3R1 RNA sample from a pool of normal or control patients. Each dot represent an mRNA sample from normal/normal pool or tumor/normal pool.

### 2. Results

Increased levels of SLC9A3R1 mRNA were detected in tumor samples obtained patients suffering from breast cancer compared to normal subjects (FIG. 1). Tumor samples from patients suffering from breast cancer averaged about 5 times higher levels of SLC9A3R1 mRNA expression than found in normal subjects (FIG. 1).

### Example 3

### Real-Time PCR Analysis of Samples Isolated from Breast Cancer Patients and Normal Breast Subjects

### 1. Patient Samples and RNA Isolation

Total RNA extraction from tumor cell lines and patient samples as described in Example 1.

### 2. Real-Time PCR

Briefly, 500 ng of total RNA was mixed with 250 μg of pdN₆ random primers (GE Healthcare, Piscataway, N.J.), and 10 pg of Arabidopsis RNA, followed by 10 min incubation at 65° C. Samples were then cooled on ice for 2 min, and mixed with the cDNA synthesis solution to final concentrations of 50 mM Tris-HCl, pH 8.3, 75 mM KCL, 3 mM MgCL₂, 10 mM DTT, 1 nM dNTP (Roche Diagnostics, Laval, QC, Canada), and 200 units of Superscript III RT enzyme (Invitrogen Corp., Carlsbad, Calif.). The samples were then incubated at 25° C. for 5 min, and 90 min. at 50° C. As a reaction control, 10 pg of RNA from Arabidopsis was added to each sample. When amplified by real-time PCR, the specific arabidopsis gene is expressed at a known levels (Ct between 19 and 20), and therefore ensures that all RT reactions worked the same. That prevents the usage of a housekeeping gene to control for the amount of cDNA. For each sample, a null RT reaction was also performed (i.e., omitting the Superscript III enzyme). This ensures that no genomic DNA was present in the total RNA preparations.

The Applied Biosystem Taqman® probes system (Foster City, Calif.), and the Light Cycler 480 (Roche Diagnostics, Laval, QC, Canada) were used for this study. The reactions were prepared as follows: 10 μl Master Mix (final concentration of 1×), 1 μl Taqman® probe (final concentration of 1×), 4 μl of Rnase/Dnase-free water (Ambion, Streetsville, ON, Canada), and 5 μl of cDNA or 5 μl of water (for No Template Control reactions) were added to each well for a final volume of 20 μl. As a reference sample, a calibrator was prepared from H460 cell lines and A549 cell lines. This calibrator was used in each experiment, and the ratios to calibrator were calculated. This allows us to compare different experiments. In each test, duplicate wells were used for different controls to ensure that all reactions were reliable. Indeed, No Template Controls and No RT controls were included, an Arabidopsis gene was amplified, and a calibrator sample was used to examine for consistency and accuracy.

The delta-delta Ct calculation method was used to analyze the real-time PCR data. In this method, the cDNA synthesis and mRNA level were normalized with a calibrator H460 and A549. Briefly, the ddct calculation compares the target gene Ct of each sample to the Ct of the calibrator with the same gene. This gives us the ratio to calibrator and allows for comparison of the samples between experiments. The calibrator also accounts for the quality of the real-time experiment as it is always expressed at the same level in all genes tested.

Levels of RNA expression are determined as ratio or folds of expression of SLC9A3R1 in each control (or normal) and tumor (test) sample relative to SLC9A3R1 RNA sample from a pool of normal or control patients. Each dot represent an mRNA sample from normal/normal pool or tumor/normal pool.

### 3. Results.

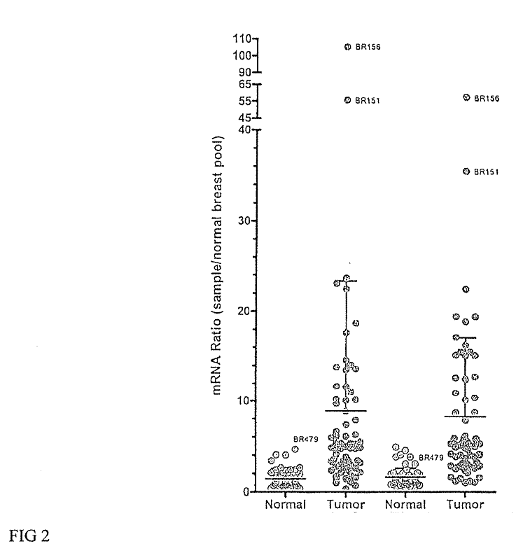

Increased levels of RNA expression was identified in breast tumor samples as compared to normal breast samples (FIG. 2). Normal breast samples showed approximately 5 times less RNA expression of SLC9A3R1 than in breast tumor samples (FIG. 2). These results confirm the results obtained from the microarray experiments shown in Example 2.

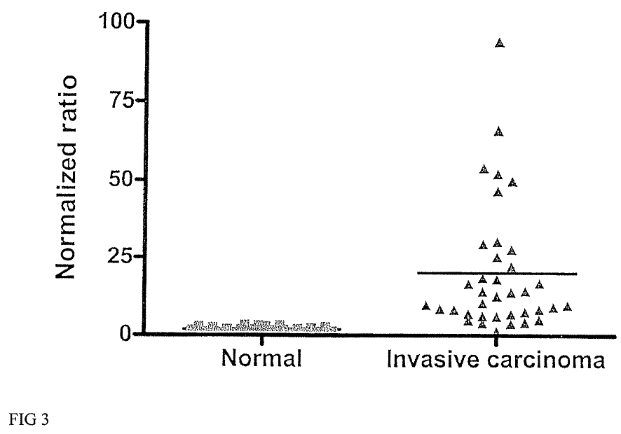

The results were also confirmed in real-time PCR studies of formalin-fixed, paraffin-embedded tissue biopsies from normal tissues and breast cancer patients (FIG. 3). Patients showed several times higher levels of SLC9A3R1 RNA as compared to the RNA expression levels found in normal subjects (FIG. 3).

### Example 4

### Preparation and Use of Focused Microarray to Detect SLC9A3R1 in Samples Obtained from Normal Lung Subjects and Lung Cancer Patients

1. Total RNA Isolation and cDNA Labeling

Patient tissues samples were obtained from Asterand, Inc. (Detroit, Mich.), Clinomics Biosciences, Inc (Watervliet, N.Y.) and Biochain Institute, Inc. (Hayward, Calif.). Each patient included in the study was screened against the same normal total RNA pool in order to compare them together. The tumor pool was composed of 11 cases. The lung normal pool was composed of 15 cases.

Patient samples, capture probes, and microarrays were prepared as described in Example 1. Fluid samples were prepared as follows. Briefly, the pleural fluid was first fractionated by centrifugation whereby both the pellet and supernatant material were mixed with lysis buffer. Protein lysates were then quantified. Microarray experiments were performed as described in Example 1. Levels of RNA expression were determined as ratio or folds of expression of SLC9A3R1 in each control (or normal) and tumor (test) sample relative to SLC9A3R1 RNA sample from a pool of normal or control patients. Each dot represents an mRNA sample from normal/normal pool or tumor/normal pool.

### 2. Results

Increased levels of SLC9A3R1 mRNA were detected in tumor fluid and cell samples obtained patients suffering from non-small lung cancer compared to the levels in fluid and cell samples obtained from normal lung subjects (FIG. 5). Tumor samples from patients suffering from breast cancer averaged about 3 to 4 times higher levels of SLC9A3R1 mRNA expression than found in normal subjects (FIG. 5). These results establish that SLC9A3R1 is a marker of neoplastic disease in lung.

### Example 5

### Real-Time PCR Analysis of Samples Isolated from Breast Cancer Patients and Normal Breast Subjects

### 1. Patient Samples and RNA Isolation

Total RNA extraction from tumor cell lines and patient samples was performed as described in Example 1.

### 2. Real-Time PCR

Real-time PCR and analysis of results was performed as described in Example 3.

### 3. Results.

Increased levels of RNA expression were identified in breast tumor samples compared to normal breast samples (FIG. 6). Normal breast samples showed approximately 4 times less RNA expression of SLC9A3R1 than found in breast tumor samples (FIG. 6). These results confirm the results obtained from the microarray experiments shown in Example 4.

### Example 6

### Western Blot Analysis of Samples Isolated from Breast Cancer Patients and Normal Breast Subjects

### 1. Patient Samples and Normal Samples

Patient tissue samples were obtained from Asterand, Inc. (Detroit, Mich.), Clinomics Biosciences, Inc (Watervliet, N.Y.) and Biochain Institute, Inc. (Hayward, Calif.). The samples were isolated from normal breast and breast cancer tissue, and were frozen into blocks of tissue. Protein cell extracts were then prepared from each block. Each patient included in the study was screened against the same normal total RNA pool in order to compare them together. The tumor pool composed of 72 cases. The breast normal pool was composed of 27 cases.

### 2. Western Blot Analysis of SLC9A3R1 in Breast Cancer and Breast Normal Samples

For breast cell samples, human tissues were homogenized using a Polytron PT10-35 (Brinkmann, Mississauga, Canada) for 30 seconds at speed setting of 4 in the presence of 300 μl of 10 mM HEPES-Tris, pH 7.4, 150 mM NaCl, 1% Triton X-100, 1% sodium deoxycholic acid, 0.1% SDS, 1 mM EDTA and a cocktail of protease inhibitors from Roche Corp. (Laval, Qc, Canada). 40 μg of proteins from human ovarian cancer patients and normal ovarian subjects were used in SDS-PAGE gels. Samples were mixed with Laemmli buffer (250 mM Tris-HCl, pH 8.0, 25% (v/v) b-mercaptoethanol, 50% (v/v) glycerol, 10% (w/v) SDS, 0.005% (w/v) bromophenol blue), heated for 5 mins. at 95° C. and resolved in 12% SDS-polyacrylamide gels (SDS-PAGE). Proteins were then electro-transferred onto Hybond-ECL nitrocellulose membranes (Amersham Biosciences, Baie d'Urfé, Canada) for 90 mins. at 100 volts at RT (RT). Membranes were blocked for 1 hr. at RT in blocking solution (PBS containing 5% fat-free dry milk). Membranes were washed with PBS and incubated with the primary anti-SLC9A3R1 polyclonal antibodies or monoclonal antibodies at the appropriate dilutions in blocking solution containing 0.02% sodium azide for 2 hrs. at RT. Antibodies were produced in house. PBS washing was performed, and the membranes were subsequently incubated for 1 hr. at RT with secondary anti-mouse, anti-rabbit or anti-goat antibodies labeled with horseradish peroxydase (Bio-Rad, Mississauga, Canada) diluted 1/3000 in PBS. Chemiluminescence detection was performed using the SuperSignal West Pico Chemiluminescent Substrate (Pierce, Rockford, Ill., USA) following the manufacturer's recommendations.

The results of expression analyses for the protein markers are shown in FIG. 7. SLC9A3R1 expression was significantly increased in tumor samples obtained from breast tumor patients as compared to expression in normal samples isolated from normal subjects. All normal subjects showed between undetectable and very low levels of SLC9A3R1 protein expression, while nearly 85% of samples obtained from breast cancer patients showed detectable levels of SLC9A3R1 (FIG. 7).

### Example 7

### ELISA Analysis of SLC9A3R1 in Breast Cancer and Breast Normal Tissues

### 1. Isolation and Preparation of Patient and Normal Tissues

Patient tissue samples were obtained and prepared as described in Example 6.

### 2. ELISA Analysis

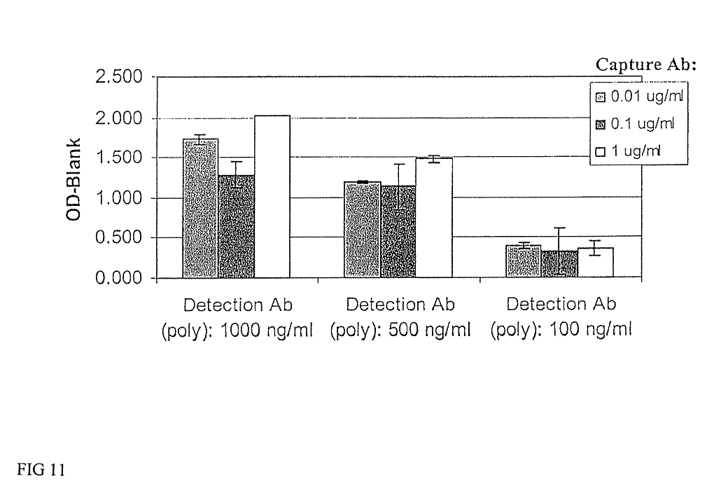

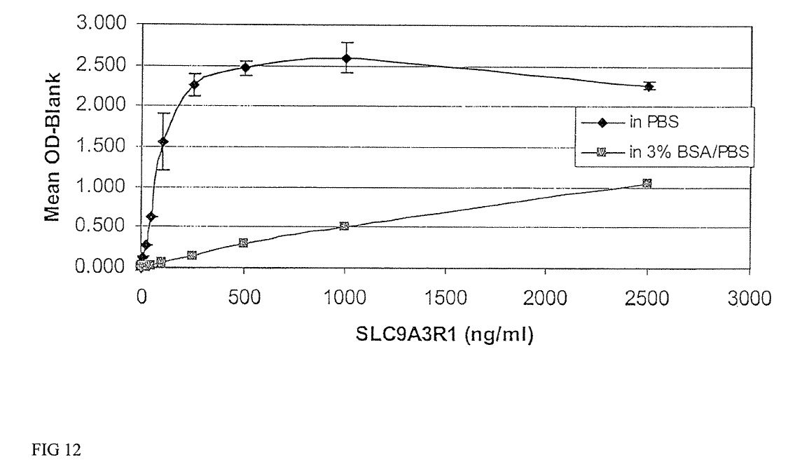

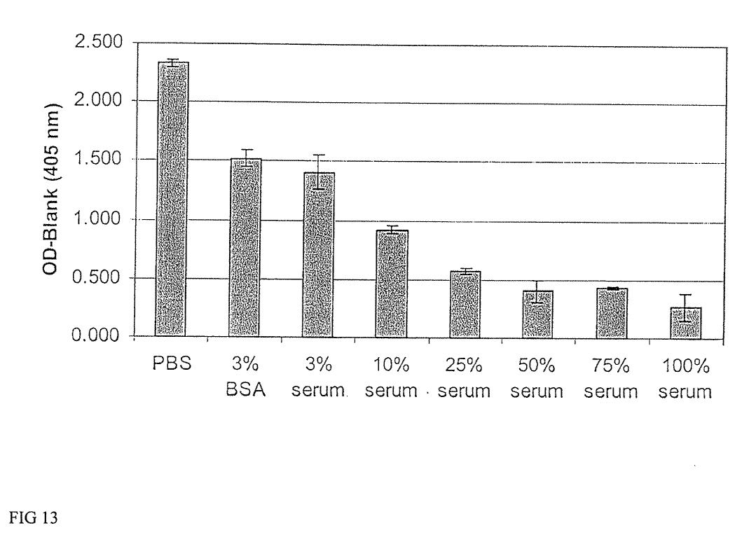

To quantify the amount of each target of interest and to confirm the results obtained by Western blot, an ELISA technique was performed on ovarian samples for SLC9A3R1. Prior to screening all samples, an optimization of the conditions was performed using normal and tumor samples to determined the linearity of the assay (dose-dependant curve, time of development of the assay). Once conditions were optimized (FIGS. 11-13), 96-well plates ((Maxisorp plates, NUNC, (Rochester, N.Y., USA)) were coated with the capture antibody. Samples were then incubated overnight at 4° C. Wells were washed 3 times with PBS and then blocked with bovine serum albumin (BSA)/PBS or BSA alone for 1 hr. RT. Detection antibodies (40 ng/well) were added to the wells and incubated for 2 hrs. RT. Plates were washed 3 times with PBS and the secondary anti-mouse, anti-rabbit or anti-goat antibodies labeled with horseradish peroxidase (Bio-Rad, Mississauga, Canada), diluted 1:3000 in 3% BSA/PBS, was incubated for 1 hr. RT. Wells were washed 3 times with PBS and developed with 2,2′-azino-bis(3-ethylbenzthiazoline-6-sulfonic acid) (ABTS) as the substrate (Sigma Corp., St. Louis, Mo.).

The intensity of the signal was assessed by reading the plates at a 405 nm wavelength using a microplate reader. For each of the target, a standard curve was established with a recombinant or purified protein at the same time to quantify the target in each sample. Results were expressed as concentrations of a target in 1 μg of total protein extract. All samples were quantified in the same assay. Differences among normal and tumor groups were analyzed using Student's two-tailed t test with significance level defined as P<0.05.

### 3. Results.

ELISA results shown in FIG. 8 are scatter plots delineating the levels of protein expression. Results are shown as ng/μg of protein marker in each normal subject versus ng/μg of protein marker in each breast cancer patient. These results confirm the results obtained in the Western blot protein analysis.

### Example 8

### Western Blot Analysis of Samples Isolated from Ovarian Cancer Patients and Normal Ovarian Subjects

### 1. Patient Samples and Normal Samples

Patient tissue samples were obtained from Asterand, Inc. (Detroit, Mich.), Clinomics Biosciences, Inc (Watervliet, N.Y.) and Biochain Institute, Inc. (Hayward, Calif.). The samples were isolated from normal ovaries and ovarian cancer tissues, and were frozen into blocks of tissue. Protein cell extracts were then prepared from each block. Each patient included in the study was screened against the same normal total RNA pool in order to compare them together. The tumor pool composed of 36 cases. The ovarian normal pool was composed of 34 cases.

### 2. Western Blot Analysis of SLC9A3R1 in Ovarian Cancer and Ovarian Normal Samples

For ovarian cell samples, human tissues were homogenized using a Polytron PT10-35 (Brinkmann, Mississauga, Canada) for 30 seconds at speed setting of 4 in the presence of 300 μl of 10 mM HEPES-Tris, pH 7.4, 150 mM NaCl, 1% Triton X-100, 1% sodium deoxycholic acid, 0.1% SDS, 1 mM EDTA and a cocktail of protease inhibitors from Roche Corp. (Laval, Qc, Canada). 40 μg of proteins from human ovarian cancer patients and normal ovarian subjects were used in SDS-PAGE gels. Samples were mixed with Laemmli buffer (250 mM Tris-HCl, pH 8.0, 25% (v/v) b-mercaptoethanol, 50% (v/v) glycerol, 10% (w/v) SDS, 0.005% (w/v) bromophenol blue), heated for 5 mins. at 95° C. and resolved in 12% SDS-polyacrylamide gels (SDS-PAGE). Proteins were then electro-transferred onto Hybond-ECL nitrocellulose membranes (Amersham Biosciences, Baie d'Urfé, Canada) for 90 mins. at 100 volts at RT (RT). Membranes were blocked for 1 hr. at RT in blocking solution (PBS containing 5% fat-free dry milk). Membranes were washed with PBS and incubated with the primary anti-SLC9A3R1 polyclonal antibodies or monoclonal antibodies at the appropriate dilutions in blocking solution containing 0.02% sodium azide for 2 hrs. at RT. Antibodies were produced in house. PBS washing was performed, and the membranes were subsequently incubated for 1 hr. at RT with secondary anti-mouse, anti-rabbit or anti-goat antibodies labeled with horseradish peroxydase (Bio-Rad, Mississauga, Canada) diluted 1/3000 in PBS. Chemiluminescence detection was performed using the SuperSignal West Pico Chemiluminescent Substrate (Pierce, Rockford, Ill., USA) following the manufacturer's recommendations.

### 3. Results.

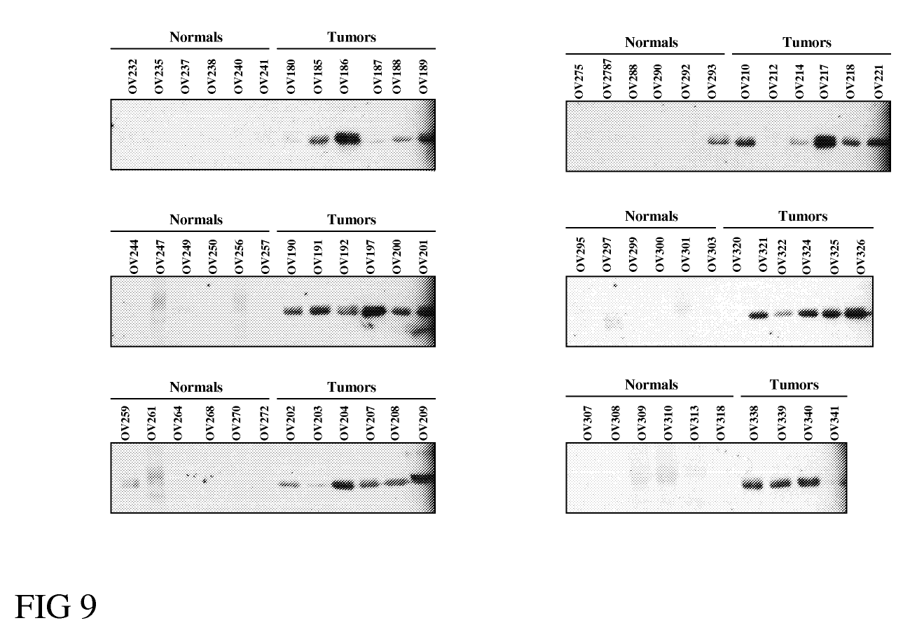

The results of expression analyses for the protein markers are shown in FIG. 9. SLC9A3R1 expression was significantly increased in tumor samples obtained from ovarian tumor patients as compared to expression in samples from normal subjects. All normal subjects showed nearly undetectable levels of SLC9A3R1 protein expression, while nearly 60% of samples obtained from ovarian cancer patients showed detectable levels of SLC9A3R1 (FIG. 9).

### Example 9

### Western Blot Analysis of Samples Isolated from Lung Cancer Patients and Normal Lung Subjects

### 1. Patient Samples and Normal Samples

Patient lung tissues and pleural fluid samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA pool in order to compare them together.

### 2. Western Blot Analysis of SLC9A3R1 in Lung Cancer and Lung Normal Samples

Fluid samples are prepared by in one of two ways: a) mixing total unfractionated pleural fluid with lysis buffer as described below; or b) the pleural fluid is first fractionated by centrifugation where both the pellet and supernatant material are mixed with lysis buffer. Protein lysates from a) and b) are then quantified and equal amounts of protein are resolved on SDS-PAGE and Western blotting.

For lung cell samples, human tissues are homogenized using a Polytron PT10-35 (Brinkmann, Mississauga, Canada) for 30 secs. at speed setting of 4 in the presence of 300 μl of 10 mM HEPES-Tris, pH 7.4, 150 mM NaCl, 1% Triton X-100, 1% sodium deoxycholic acid, 0.1% SDS, 1 mM EDTA and a cocktail of protease inhibitors from Roche Corp. (Laval, Qc, Canada).

40 μg of proteins from human lung tissue samples and fluid samples isolated from cancer patients and normal lung subjects are used in SDS-PAGE gels. Samples are mixed with Laemmli buffer, heated for 5 mins. at 95° C., and then resolved by 12% SDS-PAGE. Proteins are then electro-transferred onto Hybond-ECL nitrocellulose membranes (Amersham Biosciences, Baie d'Urfé, Canada) for 90 mins. at 100 volts at RT. Membranes are blocked for 1 hr. at RT in blocking solution (PBS containing 5% fat-free dry milk). Membranes are washed with PBS and are incubated with the primary anti-SLC9A3R1 antibodies at the appropriate dilutions in blocking solution containing 0.02% sodium azide for 2 hrs. at RT. PBS washing is performed, and the membranes are subsequently incubated for 1 hr. at RT with secondary anti-mouse, anti-rabbit or anti-goat antibodies labeled with horseradish peroxydase (Bio-Rad, Mississauga, Canada) diluted 1/3000 in PBS. Chemiluminescence detection is performed using the SuperSignal West Pico Chemiluminescent Substrate (Pierce, Rockford, Ill., USA) following the manufacturer's recommendations.

### 3. Results.

SLC9A3R1 expression is significantly increased in cell and fluid samples obtained from lung tumor patients as compared to expression in cell and fluid samples isolated from normal subjects. All normal subjects show undetectable or nearly undetectable levels of SLC9A3R1 protein expression, while samples obtained from lung cancer patients show detectable levels or increased levels of SLC9A3R1, as compared to samples from normal subjects.

### Example 10

### Western Blot Analysis of Samples Isolated from Colon Cancer Patients and Normal Colon Subjects

### 1. Patient Samples and Normal Samples

Patient tissue samples are obtained from Asterand, Inc. (Detroit, Mich.), Clinomics Biosciences, Inc (Watervliet, N.Y.) and Biochain Institute, Inc. (Hayward, Calif.). The samples are isolated from normal colon and colon cancer samples, and are frozen into blocks of tissue. Protein cell extracts are then prepared from each block. Each patient included in the study is screened against the same normal total RNA pool in order to compare them together. The tumor pool is composed of at least 20 cases. The colon normal pool is composed of at least 20 cases.

2. Western Blot Analysis of SLC9A3R1 in Colon cancer and Colon Normal Samples

Colon cell samples are isolated as described in Example 9. Western blot experiments are also performed as described in Example 9.

### 3. Results.

SLC9A3R1 expression is significantly increased in tumor samples obtained from colon tumor patients as compared to normal samples isolated from normal subjects. All normal subjects show undetectable or nearly undetectable levels of SLC9A3R1 protein expression, while samples obtained from lung cancer patients show detectable levels or increased levels of SLC9A3R1, as compared to samples from normal subjects.

### Example 111

### ELISA Analysis of SLC9A3R1 in Colon Cancer and Colon Normal Tissues

### 1. Isolation and Preparation of Patient and Normal Tissues

Patient tissue samples are obtained and are prepared as described in Example 6.

### 2. ELISA Analysis

ELISA analysis is performed as described in Example 7.

ELISA results show that samples from normal subjects expressed less SLC9A3R1 protein compared to colon cancer patient samples. These results confirm the results obtained in the Western blot expression.

### Example 12

### Preparation and Use of Focused Microarray to Detect SLC9A3R1 in Samples Obtained from Normal Colon Subjects and Colon Cancer Patients

1. Total RNA Isolation and cDNA Labeling

Patient colon tissue samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA pool in order to compare them together.

For colon tissue samples, cell lysis and RNA extraction are carried out with the RNEasy kit, (# 74104) (Qiagen, Inc., Valencia, Calif.) following the manufacturer's protocol. RNA is quantified by spectrophotometry using an Ultrospec 2000 spectrophotometer (Amersham-Biosciences, Corp., Piscataway, N.J.). RNA samples are dissolved in 10 mM Tris, pH 7.5, to determine the A_{260/280} ratios. Samples with ratios between 1.9 and 2.3 are kept for probe preparation, while samples with ratios lower than 1.9 are discarded. RNA samples are dissolved in 1 μl DEPC-H₂O for total nucleic acid quantification. Total RNA from control and treated samples is dried by speed vacuum using a Heto Vacuum centrifuge system (KNF Neuberger, Inc., Trenton, N.J.) at varying time intervals. The total RNA is resuspended in 10 μl of DEPC-H₂O and is stored at −20° C. until the labeling reaction.

First strand cDNA labeling is accomplished using 1-15 μg total RNA (depending on the cell lines to be tested) for normal and tumor cells separately. Total RNA is incubated with 4 ng control positive Arabidopsis thaliana RNA, 3 μg of Oligo (dT)₁₂₋₁₈ primer (# Y01212) (Invitrogen, Corp., Carlsbad, Calif.), 1 μg PdN6 random primer (Amersham, #272166-01) for 10 min. at 65° C., and immediately put on ice for 1 min. The mixture is then diluted in 5×First strand buffer (250 mM Tris-HCl, pH 8.3; 375 mM KCl; 15 mM MgCl₂) containing 0.1 M DTT, 0.5 μM dNTPs mix (dTTP, dGTP, dATP) (Invitrogen, #10297-018), 0.05 μM dCTP (Invitrogen, #10297-018), 5 μM Cy3-dCTP (#NEL 576) (NEN Life Science/Perkin Elmer, Boston, Mass.), 2.5 μM Cy5-dCTP (#NEL 577) (NEN Life Science/Perkin Elmer, Boston, Mass.) and 400 units SuperScript III RNAse H⁻ RT (Invitrogen, #I 8064-014). After incubating the reaction mixture for 5 min. at 25° C., the reaction mixture is incubated at 42° C. for 90 min. Finally, a total of 400 units of SuperScript II RNAse H⁻ RT (Invitrogen, #18064-014) are added and the reaction is incubated at 42° C. for another 90 min.

Digestion of the labeled cDNA with 5 units RNAse H (#M0297S) (NEB, Beverly, Mass.) and 40 units RNAse A (Amersham, # 70194Y) is done at 37° C. for 30 min. The labeling probe is purified with the QIAquick PCR purification kit (Qiagen, Inc.) protocol with some modifications. Briefly, the reaction volume is completed to 50 μl with DEPC-H₂O and 2.7 μl of 12 M NaOAc pH 5.2 is added. The reaction is diluted with 200 μl PB buffer, is put on the purification column, is spun 15 sec. at 10 000 g, is followed by 3 washes of 500 μl PE buffer (15 sec.; 10 000 g) and is eluted 2 times in 50 μl DEPC-H₂O total (1 min., 10 000 g). Frequency of incorporation and amount of cDNA labeled produced are evaluated for both labeled dCTPs by spectrophotometer (Ultrospec 2000, Pharmacia Biotech) at A₂₆₀ nm, A₅₅₀ nm and A₆₅₀ nm. The labeling material is dried by speed vacuum (Heto Vacuum centrifuge system, LaboPort) and is resuspended in 3.75 μl H₂O total for both Cy5 (normal) and Cy3 reactions (tumor).

### 2. Capture Probe Preparation

Capture probes, approximately 68 nucleotides in length corresponding to targets of interest are designed using sequences showing less identity base to base (<30%) with other coding sequences (cds) submitted to NCBI bank. The comparisons between sequences are done by BLAST research (www.ncbi.nlm.nih.gov/BLAST). For BioChip ver1.0 and ver2.0, a basic melting point temperature at a salt concentration of 50 mM Na⁺ (Tm) for each capture probe is calculated: the overall average is 76.97° C.+/−3.72° C. GC nucleotide content averaged 51.2%+/−9.4%. For the present invention, two negative controls (68 bp of the antisense cds of the BRCP and nucleophosmin targets) are synthesized.

The SLC9A3R1 nucleic acid capture probe targets SLC9A3R1 GenBank Accession No. NM_{—}004252 (SEQ ID NO: 1). CRAB-PII nucleic acid capture probe targets GenBank Accession No. NM_{—}001878 (SEQ ID NO: 2). Enolase-1 nucleic acid capture probe targets GenBank Accession No. NM_{—}001428 (SEQ ID NO: 3). Cytokeratin 18 nucleic acid capture probe targets GenBank Accession No. NM_{—}000224 (SEQ ID NO: 4). Triosephosphate isomerase nucleic acid capture probe targets GenBank Accession No. U47924 (SEQ ID NO: 5). Stratifin nucleic acid capture probe targets GenBank Accession No. NM_{—}006142 (SEQ ID NO: 6). HPRT nucleic acid capture probe targets GenBank Accession No. NM_{—}000194 (SEQ ID NO: 7). Cytokeratin 18 nucleic acid capture probe targets GenBank Accession No. NM_{—}199187 (SEQ ID NO: 8).

The capture probe is synthesized by the BRI Institute (Biotechnology Research Institute, Clear Water Bay, Kowloon, Hong Kong, China) with the Expedilite™ Synthesizer (Applied Biosystems, Foster City, Calif.). The oligonucleotides are verified by PAGE. Oligonucleotide quantification is done by spectrophotometry at A_{260 nm}.

### 3. Printing of Capture Probes and Production of the Focused Microarray

Prior to printing of capture probes, different dilutions of Arabidopsis thaliana chlorophyll synthetase G4 DNA (undiluted solutions at 0.15 μg/μl and at 0.2 μg/μl; 1:2; 1:4; 1:8; 1:16) are printed on each grid as a positive control, and for normalization of results. Preparation of Arabidopsis thaliana control capture probes is performed as follows. Briefly, five micrograms of a Midi preparation using a HiSpeed™ Plasmid Midi kit (Qiagen, Inc.) of the Arabidopsis thaliana plasmid (gift of BRI) is digested with 40 units of Sac I enzyme (NEB) for 2 hr. at 37° C., is purified with the QIAquick PCR purification kit (Qiagen,) and is verified by 1% agarose migration. In vitro transcription of 2 μg Sac I digestion is performed in 10×transcription buffer (400 mM Tris-HCl, pH 8.0; 60 mM MgCl₂; 100 mM DTT; 20 mM Spermidin) containing 2 μl of 10 mM NTP mix (Invitrogen), 20 units RNAse OUT (Invitrogen, #10777-019) and 50 units T7 RNA polymerase (NEB) for approximately 2 hr. to 30 hr. at 37° C. The reaction is then treated with 2 units DNAse I (Invitrogen) in 10×DNAse buffer (200 mM Tris-HCl pH 8.4; 20 mM MgCl₂; 500 mM KCl) for 15 min. at 37° C. The RNA is cleaned with the RNEasy kit (Qiagen) and is quantified by spectrophotometry using an Ultrospec 2000 (Amersham Biosciences, Corp.).

After the control capture probes are generated and printed, the capture probes complementary to marker genes from the cancer cell samples are printed at concentrations of 25 μM in 50% DMSO on CMT-GAPS II Slides (# 40003) (Corning, 45 Nagog Park, Acton, Mass.) by the VersArray CHIP Writer Prosystems (BioRad Laboratories) with the Stealth Micro Spotting Pins (#SMP3) (Telechem International, Inc., Sunnyvale, Calif.). Each capture probe is printed in triplicate on duplicate grids. Buffer and Salmon Testis DNA (Sigma D-7656) are also printed for the BioChip analysis step. After printing is completed, the slides are dried overnight by incubation in the CHIP Writer chamber. Chips are then treated by UV (Stratagene, UV Stratalinker) at 600 mJoules and are baked in an oven for 6-8 hr.

### 4. Quality Control of Focused Microarray

Prior to testing the invention on cancer cell samples, the focused microarray is tested at the BRI Institute (Kowloon Bay, Hong Kong). One slide for each printed batch is quality control tested using a terminal deoxynucleotidyl transferase (Tdt)-mediated nick end labeling assay protocol (see, e.g., Yeo et. al., (2004) Clin. Cancer Res. 10(24): 8687-96). Additionally, controls are performed to verify the specificity of the hybridization using three independent grids on the same focused microarray.

As a first quality control, a test is done by the BRI Institute on one slide for each batch printed with the following Tdt transferase protocol. Briefly, the slide is prehybridized in a Hybridization Chamber (#2551) (Corning, Inc., Life Sciences, 45 Nagog Park, Acton, Mass.) with 80 μl of preheated prehybridization buffer (5×SSC (750 mM NaCl; 75 mM sodium citrate); 0.1% SDS; 1% BSA (Sigma, #A-7888) at 37° C. for 30 min. Slides are washed in 0.1×SSC (15 mM NaCl; 1.5 mM sodium citrate) and are air-dried. 50 μl of TdT reaction mixture [5×TdT buffer (125 mM Tris-HCl, pH 6.6, 1 M sodium cacodylate, 1.25 mg/ml BSA); 5 mM CoCl₂; 1 mM Cy3-dCTP (NEN Life Science, NEL 576); 50 units TdT enzyme (#27-0730-01) (Amersham BioSciences)], is added to the entire area of the BioChip. The slide is incubated in the Hybridization Chamber for 60 min. at 37° C. following by a first wash in 1×SSC (150 mM NaCl; 15 mM sodium citrate)/0.2% SDS (preheated at 37° C.) for 10 min., a second wash of 5 min. in 0.1×SCC (15 mM NaCl; 1.5 mM sodium citrate)/0.2% SDS at RT and finally a last wash of 5 min. at RT in 0.1×SSC (15 mM NaCl; 1.5 mM sodium citrate). The slide is scanned with the ScanArray™ Lite MicroArray Scanner (Packard BioSciences, Perkin Elmer, San Jose, Calif.).

As a second quality control step, the PARAGON™ DNA Microarray Quality Control Stain kit (Molecular Probes) is incubated with the microarray according to the manufacturer's recommendations.

5. Focused Microarray Hybridization with Labeled cDNA Probes

Focused microarray slides are pre-washed before the prehybridization step as follows. First, slides are washed for 20 min. at 42° C. in 2×SSC (300 mM NaCl; 30 mM sodium citrate)/0.2% SDS under agitation. The second wash is for 5 min. at RT in 0.2×SSC (30 mM NaCl, 3 mM sodium citrate) under agitation, and then is followed by a wash for 5 min. at RT in DEPC-H₂O with agitation. The slides are spin dried at 1000 g for 5 min. and prehybridized in Dig Easy Hyb Buffer (#1,603,558) (Roche Diagnostics Corporation, Indianapolis, Ind.) containing 400 μg Bovine Serum Albumin (Roche, #711,454) at 42° C. in humid chamber for 3 hrs. The chips are then washed 2 times in DEPC-H₂O, and once in Isopropanol (Sigma, 1-9516) and are spun dry at 1000 g for 5 min.

To the mixed Cy5/Cy3 probe, 15 μg Baker tRNA (#109,495) (Roche Diagnostics Corp., Indianapolis, Ind.) and 1 μg Cot-1DNA (Roche, #1,581,074) are added and the probe is incubated 5 min. at 95° C., put on ice for 1 min., and diluted with 14 μl Dig Easy Hyb buffer (Roche, #1,603,558). After a 2 min. spin at 100 g, the probe is incubated at 42° C. for at least 5 min.

The three supergrids on the slide are separated by a Jet-Set Quick Dry TOP Coat 101 line (#FX268) (L'Oreal, Paris, FR). Each probe is added to its respective supergrid and is covered by a preheated (42° C.) coverslip (Mandel, #S-104 84906). The slide is incubated at 42° C. in humid chamber for at least 15 hrs.

The coverslips are removed by dipping in 1×SSC (150 mM NaCl; 15 mM sodium citrate)/0.2% SDS solution preheated at 50° C.). The slide is washed three times for 5 min. with agitation in 1×SSC (150 mM NaCl; 15 mM sodium citrate)/0.2% SDS solution preheated at 50° C.), and is then washed three times with agitation in 0.1×SSC (15 mM NaCl; 1.5 mM sodium citrate)/0.2% SDS solution preheated at 37° C.). Finally, the slide is washed once in 0.1×SSC (15 mM NaCl; 1.5 mM sodium citrate) with agitation for 5 min. The slide is dipped several times in DEPC-H₂O and spun dry at 1000 g for 5 min.

### 6. Scanning and Statistical Analysis

The slides are scanned with a ScanArray® Lite MicroArray Scanner (Packard BioSciences, Perkin Elmer, San Jose, Calif.) and the analysis is performed with a QuantArray® Microarray Analysis software version 3.0 (Packard BioSciences, Perkin Elmer, San Jose, Calif.).

The QuantArray® data results are analyzed according to the procedures described above in Example 2(6).

### 7. Results

SLC9A3R1 mRNA expression correlates with SLC9A3R1 protein expression. Increased levels of SLC9A3R1 mRNA are detected in samples obtained from patients suffering from colon cancer as compared to that in normal subjects. Cell samples from patients suffering from colon cancer have higher levels of SLC9A3R1 mRNA expression than did samples from normal subjects.

### Example 13

### Real-Time PCR Analysis of Samples Isolated from Colon Cancer Patients and Normal Colon Subjects

### 1. Patient Samples and RNA Isolation

Total RNA extraction from tumor cell lines and patient samples is performed as described in Example 5.

### 2. Real-Time PCR

Real-time PCR and analysis of results is performed as shown in Example 3.

### 3. Results.

Increased levels of RNA expression are identified in colon tumor samples as compared to expression in normal colon samples. Normal colon samples show less RNA expression of SLC9A3R1 than do colon tumor samples. These results confirm the results obtained from the microarray experiments described in Example 12.

### Example 14

### Western Blot Analysis of Samples Isolated from Prostate Cancer Patients and Normal Prostate Subjects

### 1. Patient Samples and Normal Samples

Patient tissue samples are obtained from Asterand, Inc. (Detroit, Mich.), Clinomics Biosciences, Inc (Watervliet, N.Y.) and Biochain Institute, Inc. (Hayward, Calif.). The samples are isolated from normal prostate and prostate cancer samples, and are frozen into blocks of tissue. Protein cell extracts are then prepared from each block. Each patient included in the study is screened against the same normal total RNA pool in order to compare them together. The tumor pool is composed of at least 20 cases. The Prostate normal pool is composed of at least 20 cases.

### 2. Western Blot Analysis of SLC9A3R1 in Prostate Cancer and Prostate Normal Samples

Patient and normal samples are performed as described in Example 9. Western blot analysis is also performed as described in Example 9.

### 3. Results.

SLC9A3R1 expression is significantly increased in samples obtained from prostate tumor patients compared to samples isolated from normal subjects. All normal subjects show undetectable or nearly undetectable levels of SLC9A3R1 protein expression, while samples obtained from lung cancer patients show detectable levels or increased levels of SLC9A3R1, as compared to samples from normal subjects.

### Example 15

### ELISA Analysis of SLC9A3R1 in Prostate Cancer and Normal Prostate Tissues

### 1. Isolation and Preparation of Patient and Normal Tissues

Patient tissue samples are obtained and are prepared as described in Example 6.

### 2. ELISA Analysis

ELISA analysis is performed as described in Example 7.

### 3. Results.

ELISA results show that normal samples express less SLC9A3R1 protein compared to prostate cancer samples. These results confirm the results obtained in the Western blot expression.

### Example 16

### Preparation and use of the Focused Microarray To Detect SLC9A3R1 in Samples Obtained from Normal Prostate Subjects and Prostate Cancer Patients

1. Total RNA Isolation and cDNA Labeling

Patient prostate tissue samples are obtained from Asterand, Inc. (Detroit, Mich.), Clinomics Biosciences, Inc (Watervliet, N.Y.) and Biochain Institute, Inc. (Hayward, Calif.). Each patient that is included in the study is screened against the same normal total RNA pool in order to compare them together.

### 2. Capture Probe Preparation, Preparation of Focused Microarray, Quality Control Hybridization and Analysis

Capture probe preparation and printing of capture probes are performed according to the procedure provided in Example 12. The preparation of the microarray, quality control, hybridization, and analysis of the results are performed as detailed in Example 12.

### 3. Results

SLC9A3R1 mRNA expression correlates with SLC9A3R1 protein expression. Increased levels of SLC9A3R1 mRNA are detected in cell samples obtained patients suffering from prostate cancer compared to samples from normal subjects. Cell samples from patients suffering from prostate cancer have higher levels of SLC9A3R1 mRNA expression than normal subjects.

### Example 17

### Real-Time PCR Analysis of Samples Isolated from Prostate Cancer Patients and Normal Prostate Subjects

### 1. Patient Samples and RNA Isolation

Total RNA extraction from tumor cell lines and from patient samples is performed as described in Example 5.

### 2. Real-Time PCR

Real-time PCR and analysis of results is performed as shown in Example 3.

### 3. Results.

Increased levels of RNA expression are identified in prostate tumor samples compared to normal colon samples. Normal prostate samples show less RNA expression of SLC9A3R1 than prostate tumor samples. These results confirm the results obtained from the microarray experiments shown in Example 16.

### Example 18

### Western Blot Analysis of Samples Isolated from Leukemia Patients and Normal Subjects

### 1. Patient Samples and Normal Samples

Patient marrow tissues and blood are obtained from Asterand, Inc. (Detroit, Mich.), Clinomics Biosciences, Inc (Watervliet, N.Y.) and Biochain Institute, Inc. (Hayward, Calif.). Each patient sample included in the study is screened against the same normal total RNA pool in order to compare them together.

### 2. Western Blot Analysis of SLC9A3R1 in Leukemia and Normal Samples

Blood samples are prepared by isolating blood from leukemia patients. The blood samples are fractioned initially to isolate remove red-blood cells. The samples containing all white blood cell are further fractionated by FACS sorting based on size defractions and/or using surface specific monoclonal antibodies. Purified cells are then lysed in lysis buffer as described in the above examples. Quantified cell lysates from leukemia samples and normal blood cells are then resolved on SDS-PAGE and prepared for Western blotting to probe for SLC9A3R1 and other biomarkers.

### 3. Results.

SLC9A3R1 expression is significantly increased in cell and fluid samples obtained from tumor patients as compared to expression in cell and fluid samples isolated from normal subjects. All normal subjects show undetectable or nearly undetectable levels of SLC9A3R1 protein expression, while samples obtained from lung cancer patients show detectable levels or increased levels of SLC9A3R1, as compared to samples from normal subjects.

### Example 19

### Preparation and Use of Focused Microarray to Detect SLC9A3R1 in Samples Obtained From Normal Subjects and Leukemia Patients

1. Total RNA Isolation and cDNA Labeling

Patient marrow tissues and blood are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA pool in order to compare them together.

Blood samples are prepared as described in Example 18. For leukemia tissue samples, human marrow tissues are homogenized and prepared for analysis following procedures described in Example 8.

First strand cDNA labeling, cDNA digestion, capture probe preparation and focused microarray preparation are accomplished using procedures described in Example 1. In addition, quality control and focused microarray hybridization are performed according to procedures described in Example 1. The QuantArray® data results are analyzed according to the procedures described above in Example 1.

### 2. Results.

SLC9A3R1 mRNA expression correlates with SLC9A3R1 protein expression. Increased levels of SLC9A3R1 mRNA are detected in cell and fluid samples obtained patients suffering from leukemia compared to expression in samples from normal subjects. Cell and fluid samples from patients suffering from leukemia have higher levels of SLC9A3R1 mRNA expression than do samples from normal subjects.

### Example 20

### Western Blot Analysis of Samples Isolated from Sarcoma Patients and Normal Subjects

### 1. Patient Samples and Normal Samples

Patient tissue samples are obtained from Asterand, Inc. (Detroit, Mich.), Clinomics Biosciences, Inc (Watervliet, N.Y.) and Biochain Institute, Inc. (Hayward, Calif.). The samples are isolated from normal Sarcoma and Sarcoma cancer samples, and are frozen into blocks of tissue. Protein cell extracts are then prepared from each block. Each patient included in the study is screened against the same normal total RNA pool in order to compare them together. The tumor pool is composed of at least 20 cases. The Prostate normal pool is composed of at least 20 cases.

### 2. Western Blot Analysis of SLC9A3R1 in Sarcoma Cancer and Sarcoma Normal Samples

Sample preparation and western blot analysis are performed as described in Example 9.

### 3. Results.

SLC9A3R1 expression is increased in tumor samples obtained from sarcoma tumor patients compared to expression in normal samples isolated from normal subjects. All normal subjects show undetectable or nearly undetectable levels of SLC9A3R1 protein expression, while samples obtained from lung cancer patients show detectable levels or increased levels of SLC9A3R1, as compared to samples from normal subjects.

### Example 21

### ELISA Analysis of SLC9A3R1 in Sarcoma Cancer and Sarcoma Normal Tissues

### 1. Isolation and Preparation of Patient and Normal Tissues

Patient tissue samples are obtained and are prepared as described in Example 6.

### 2. ELISA Analysis

ELISA analysis is performed as described in Example 7.

### 3. Results.

ELISA results show that samples from normal subjects expressed less SLC9A3R1 protein compared to samples from sarcoma cancer patients. These results confirm the results obtained by the Western blot analysis.

### Example 22

### Preparation and Use The Focused Microarray to Detect SLC9A3R1 in Samples Obtained From Normal Sarcoma Subjects and Sarcoma Cancer Patients

1. Total RNA Isolation and cDNA Labeling

Patient Sarcoma tissue samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA pool in order to compare them together.

### 2. Capture Probe Preparation, Preparation of Focused Microarray, Quality Control Hybridization and Analysis

Capture probe preparation and printing of capture probes are performed according to the procedure provided in Example 12. The preparation of the microarray, quality control, hybridization, and analysis of the results are performed as described in Example 12.

### 3. Results

SLC9A3R1 mRNA expression correlates with SLC9A3R1 protein expression. Increased levels of SLC9A3R1 mRNA are detected in cell sample obtained patients suffering from sarcoma cancer compared to expression in samples from normal subjects. Cell samples from patients suffering from sarcoma cancer have higher levels of SLC9A3R1 mRNA expression than do normal subjects.

### Example 23

### Real-Time PCR Analysis of Samples Isolated from Sarcoma Cancer Patients and Normal Sarcoma Subjects

### 1. Patient Samples and RNA Isolation

Total RNA extraction from tumor cell lines and patient samples is performed as described in Example 5.

### 2. Real-Time PCR

Real-time PCR and analysis of results are performed as shown in Example 3.

### 3. Results.

Increased levels of RNA expression are identified in colon tumor samples compared to normal colon samples. Normal sarcoma samples show less RNA expression of SLC9A3R1 than do sarcoma tumor samples. These results confirm the results obtained from the microarray experiments described in Example 22.

### Example 24

### Western Blot Analysis of Samples Isolated from Melanoma Patients and Normal Subjects

### 1. Patient Samples and Normal Samples

Patient tissues and fluid samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA pool in order to compare them together.

### 2. Western Blot Analysis of SLC9A3R1 in Melanoma and Normal Samples

Sample preparation and Western blot analysis are performed as described in Example 9.

### 3. Results.

SLC9A3R1 expression is increased in samples obtained from melanoma tumor patients compared to samples isolated from normal subjects. All normal subjects show undetectable or nearly undetectable levels of SLC9A3R1 protein expression, while samples obtained from melanoma cancer patients show detectable levels of SLC9A3R1.

### Example 25

### ELISA Analysis of SLC9A3R1 in Melanoma Cancer and Melanoma Normal Tissues

### 1. Isolation and Preparation of Patient and Normal Tissues

Patient tissue samples are obtained and are prepared as described in Example 6.

### 2. ELISA Analysis

ELISA analysis is performed as described in Example 7.

ELISA results show that normal subjects expressed less SLC9A3R1 protein compared to melanoma cancer patient samples. These results confirm the results obtained in the Western blot expression.

### Example 26

### Preparation and Use of Focused Microarray to Detect SLC9A3R1 in Samples Obtained From Normal Melanoma Subjects and Melanoma Cancer Patients

1. Total RNA Isolation and cDNA Labeling

Patient Melanoma tissue samples are obtained from Asterand, Inc. (Detroit, Mich.), Clinomics Biosciences, Inc (Watervliet, N.Y.) and Biochain Institute, Inc. (Hayward, Calif.). Each patient included in the study is screened against the same normal total RNA pool in order to compare them together.

### 2. Capture Probe Preparation, Preparation of Focused Microarray, Quality Control Hybridization and Analysis

Capture probe preparation and printing of capture probes are performed according to the procedure provided in Example 12. The preparation of the microarray, quality control, hybridization, and analysis of the results is performed as detailed in Example 12.

### 3. Results

SLC9A3R1 mRNA expression correlates with SLC9A3R1 protein expression. Increased levels of SLC9A3R1 mRNA are detected in cell obtained patients suffering from melanoma cancer compared to normal subjects. Cell samples from patients suffering from melanoma cancer have higher levels of SLC9A3R1 mRNA expression than would be found in samples from normal subjects.

### Example 27

### Real-Time PCR Analysis of Samples Isolated from Melanoma Cancer Patients and Normal Melanoma Subjects

### 1. Patient Samples and RNA Isolation

Total RNA extraction from tumor cell lines and patient samples is performed as described in Example 5.

### 2. Real-Time PCR

Real-time PCR and analysis of results is performed as described in Example 3.

### 3. Results.

Increased levels of RNA expression are identified in colon tumor samples compared to expression in normal colon samples. Normal melanoma samples show less SLC9A3R1 RNA expression than do melanoma tumor samples. These results confirm the results obtained from the microarray experiments described in Example 26.

### EQUIVALENTS

Those skilled in the art will recognize, or be able to ascertain, using no more than routine experimentation, numerous equivalents to the specific compositions and procedures described herein. Such equivalents are considered to be within the scope of this invention, and are covered by the following claims.

I claim:

1. A method for detecting a neoplasm comprising:
a) obtaining a potentially neoplastic test cell sample and a non-neoplastic control cell sample;
b) detecting a level of SLC9A3R1 expression in the test cell sample and in the control cell sample; and
c) comparing the level of SLC9A3R1 expression in the test cell sample to the level of SLC9A3R1 expression in the control cell sample,
wherein the test cell sample is neoplastic if the level of SLC9A3R1 expression in the test cell sample is detectably greater than the level of SLC9A3R1 expression in the control cell sample.

2. The method of claim 1, wherein detecting the level of expression of SLC9A3R1 comprises isolating a cellular cytoplasmic fraction from the test cell sample and from the control cell sample, and then separately detecting the level of expression of SLC9A3R1 in these cellular cytoplasmic fractions.

3. The method of claim 1, wherein the level of expression of SLC9A3R1 protein is detected by contacting the test cell sample and the control cell sample with a SLC9A3R1-specific protein binding agent selected from the group consisting of an anti-SLC9A3R1-specific antibodies and anti-SLC9A3R1-specific fragments thereof.

4. The method of claim 3, wherein the protein binding agent bound to SLC9A3R1 protein further comprises a detectable label selected from the group consisting of immunofluorescent label, a radiolabel, and a chemiluminescent label.

5. The method of claim 3, wherein the protein binding agent is immobilized on a solid support.

6. The method of claim 1, wherein SLC9A3R1 expression is detected by detecting the level of expression of SLC9A3R1 RNA by contacting the test cell sample and the control cell sample with a nucleic acid binding agent selected from the group consisting of RNA, cDNA, cRNA, and RNA-DNA hybrids, and further determining how much nucleic acid binding agent is hybridized to SLC9A3R1 RNA.

7. The method of claim 6, wherein the level of nucleic acid binding agent hybridized to SLC9A3R1 RNA is detected using a detectable label operably linked to the binding agent, the label being selected from the group consisting of an immunofluorescent label, a radiolabel, and a chemiluminescent label.

8. The method of claim 7, wherein the nucleic acid binding agent is immobilized on a solid support.

9. The method of claim 1, wherein the level of expression of SLC9A3R1 in the test cell sample is at least 1.5 times greater than the level of expression of SLC9A3R1 in the control cell sample.

10. The method of claim 1, wherein the level of expression of SLC9A3R1 in the test cell sample is at least 2 times greater than the level of expression of SLC9A3R1 in the control cell sample.

11. The method of claim 1, wherein the level of expression of SLC9A3R1 in the test cell sample is at least 4 times greater than the level of expression of SLC9A3R1 in the control cell sample.

12. The method of claim 1, wherein the level of expression of SLC9A3R1 in the test cell sample is at least 6 times greater than the level of expression of SLC9A3R1 in the control cell sample.

13. The method of claim 1, wherein the level of expression of SLC9A3R1 in the test cell sample is at least 8 times greater than the level of expression of SLC9A3R1 in the control cell sample.

14. The method of claim 1, wherein the level of expression of SLC9A3R1 in the test cell sample is at least 10 times greater than the level of expression of SLC9A3R1 in the control cell sample.

15. The method of claim 1, wherein the level of expression of SLC9A3R1 in the test cell sample is at least 20 times greater than the level of expression of SLC9A3R1 in the control cell sample.

16. The method of claim 1, wherein the test cell sample is isolated from a tissue of a patient suffering from a metastasized ovarian neoplastic disease, the tissue being selected from the group consisting of blood, bone marrow, spleen, lymph node, liver, thymus, kidney, brain, skin, gastrointestinal tract, eye, breast, and prostate.

17. The method of claim 1, wherein the test cell sample is isolated from a patient suffering from an ovarian neoplasm selected from the group consisting of ovarian carcinoma, ovarian epithelial adenocarcinoma, ovarian adenocarcinoma, sex cord-stromal carcinoma, endometrioid tumors, mucinous carcinoma, germ cell tumors, and clear cell tumors.

18. The method of claim 1, wherein the test cell sample is obtained from a patient suffering a breast neoplasm.

19. A method for diagnosing cancer a subject comprising:
a) obtaining a potentially neoplastic test fluid sample from the subject and a non-neoplastic control fluid sample;
b) detecting a level of SLC9A3R1 expression in the test fluid and in the control fluid
c) comparing the level of SLC9A3R1 expression in the test fluid sample to the level of SLC9A3R1 expression in the control fluid sample,
wherein cancer is diagnosed if the level of SLC9A3R1 expression in the test fluid sample is detecting greater than the level of SLC9A3R1 expression in the control fluid sample.

20. The method of claim 18, wherein detecting the level of SLC9A3R1 expression comprises isolating cellular cytoplasmic fractions from the test fluid sample and from the control fluid sample, and then detecting the level of SLC9A3R1 expression in the test and control cellular cytoplasmic fractions.

21. The method of claim 18, wherein the levels of SLC9A3R1 expression protein are determined by contacting the test fluid sample and the control fluid sample with a protein binding agent selected from the group consisting of an anti-SLC9A3R1 antibody and binding fragments thereof.

22. The method of claim 21, wherein the protein binding agent SLC9A3R1, SLC9A3R1 binding fragments of the antibody, and further comprises a detectable label selected from the group consisting of an immunofluorescent label, a radiolabel, and a chemiluminescent label.

23. The method of claim 21, wherein the protein binding agent is immobilized on a solid support.

24. The method of claim 21, wherein the level of expression SLC9A3R1 protein expression is determined by measuring the level of anti-SLC9A3R1 antibody in the test fluid sample and in the control fluid sample.

25. The method of claim 24, wherein the level of expression of anti-SLC9A3R1 antibody is detected in a serum sample isolated from a subject, potentially suffering from a neoplasm, and from a subject not suffering from a neoplasm.

26. The method of claim 25, wherein the level of expression of anti-SLC9A3R1 antibody is detected by anti-SLC9A3R1 antibody or fragments thereof.

27. The method of claim 26, wherein the anti-SLC9A3R1 antibody or binding fragments thereof are operably linked to a detectable label selected from the group consisting of a immunofluorescent label, radiolabel, and chemiluminescent label.

28. The method of claim 19, wherein SLC9A3R1 expression is measured by detecting the level of SLC9A3R1 RNA expression by contacting the test fluid sample and the non-neoplastic fluid control fluid sample with a nucleic acid binding agent selected from the group consisting of RNA, cDNA, cRNA, and RNA-DNA hybrids and determining how much nucleic acid binding agents is hybridized to SLC9A3R1 RNA.

29. The method of claim 28, wherein nucleic acid binding agent further comprises a detectable label selected from the group consisting of immunofluorescent label, radiolabel, and chemiluminescent label.

30. The method of claim 28, wherein the nucleic acid binding agent is immobilized on a solid support.

31. The method of claim 19, wherein the level of expression of SLC9A3R1 in the test fluid sample is about 1.5 times greater than the level of expression of SLC9A3R1 in the control fluid sample.

32. The method of claim 19, wherein the level of expression of SLC9A3R1 in the test fluid sample is about 2 times greater than the level of expression of SLC9A3R1 in the control fluid sample.

33. The method of claim 19, wherein the level of expression of SLC9A3R1 in the test fluid sample is about 4 times greater than the level of expression of SLC9A3R1 in the control fluid sample.

34. The method of claim 19, wherein the level of expression of SLC9A3R1 in the test fluid sample is about 6 times greater than the level of expression of SLC9A3R1 in the control fluid sample.

35. The method of claim 19, wherein the level of expression of SLC9A3R1 in the test fluid sample is about 8 times greater than the level of expression of SLC9A3R1 in the control fluid sample.

36. The method of claim 19, wherein the level of expression of SLC9A3R1 in the test fluid sample is about 10 times greater than the level of expression of SLC9A3R1 in the control fluid sample.

37. The method of claim 19, wherein the level of expression of SLC9A3R1 in the test fluid sample is at least 20 times greater than the level of expression of SLC9A3R1 in the control fluid sample.

38. The method of claim 19, wherein the test fluid sample is from a patient suffering from a metastasized neoplastic disease isolated from a tissue selected from the group consisting of blood, bone marrow, spleen, lymph node, liver, thymus, kidney, brain, skin, gastrointestinal tract, eye, breast, and prostate.

39. The method of claim 19, wherein the test fluid sample is from a patient suffering from an ovarian neoplasm selected from the group consisting of ovarian carcinoma, ovarian epithelial adenocarcinoma, ovarian adenocarcinoma, sex cord-stromal carcinoma, endometrioid tumors, mucinous carcinoma, germ cell tumors, and clear cell tumors.

40. A method for detecting a neoplasm comprising:
a) obtaining a potentially neoplastic test sample and a non-neoplastic control sample;
b) detecting a level of SLC9A3R1 expression in the test sample and in the control sample;
c) detecting a level of expression of at least one of CRAB-PII, enolase I, cytokeratine 18, triosephosphate isomerase, SFN, and/or HPRT; and
d) comparing the level of SLC9A3R1 expression and the level of expression of at least one of enolase I, cytokeratine 18, triosephosphate isomerase, SFN, and/or HPRT in the test sample to the level of SLC9A3R1 expression and the level of expression of at least one of enolase I, cytokeratine 18, triosephosphate isomerase, SFN, and/or HPRT in the control sample,
wherein the test sample is neoplastic if the levels of expression of SLC9A3R1 and at least one of enolase I, cytokeratine 18, triosephosphate isomerase, SFN, and/or HPRT in the test sample are detectably greater than the levels of expression of SLC9A3R1 and at least one of enolase I, cytokeratine 18, triosephosphate isomerase, SFN, and/or HPRT in the control sample.

41. The method of claim 40, wherein detecting the level of expression of SLC9A3R1 and the level of expression of at least one of enolase I, cytokeratine 18, triosephosphate isomerase, SFN, and/or HPRT comprises isolating a cellular cytoplasmic fraction from the test sample and from the control sample, and then detecting the levels of expression of SLC9A3R1 and at least one of enolase I, cytokeratine 18, triosephosphate isomerase, SFN, and/or HPRT in each of these cellular cytoplasmic fractions.

42. The method of claim 40, wherein the level of SLC9A3R1 expression is detected by contacting the test sample and the control sample with a SLC9A3R1-specific protein binding agent selected from the group consisting of an SLC9A3R1 specific antibody, SLC9A3R1-binding portions of an antibody, SLC9A3R1-specific ligand, an SLC9A3R1-specific aptamer, and an SLC9A3R1 inhibitor.

43. The method of claim 42, wherein the SLC9A3R1-specific protein binding agent is immobilized on a solid support.

44. The method of claim 40, wherein the level of expression of SLC9A3R1 is measured by detecting a level of anti-SLC9A3R1 antibody in a test fluid sample and in a control fluid sample.

45. The method of claim 44, wherein the test fluid sample are serum samples isolated from a subject potentially suffering from a neoplasm and from a subject not suffering from a neoplasm.

46. The method of claim 40, wherein the level of expression of SLC9A3R1 is measured by measuring the level of SLC9A3R1 RNA and the level of expression of at least one of enolase I RNA, cytokeratine 18 RNA, triosephosphate isomerase RNA, SFN RNA, and/or HPRT RNA are detected in the test sample and in the control sample.

47. The method of claim 46, wherein the level of expression of SLC9A3R1 RNA and the level of expression of at least one of enolase I RNA, cytokeratine 18 RNA, triosephosphate isomerase RNA, SFN RNA, and/or HPRT RNA are detected by contacting the test sample and the control sample with an SLC9A3R1-specific nucleic acid binding agent and with an isomerase-specific, an SFN-specific, and an HPRT-specific nucleic acid binding agent selected from the group consisting of RNA, cDNA, cRNA, and RNA-DNA hybrids.

48. The method of claim 47, wherein the nucleic acid binding agents are immobilized on a solid support.

49. The method of claim 46, wherein the level of expression of SLC9A3R1, enolase I RNA, cytokeratine 18 RNA, triosephosphate isomerase RNA, SFN RNA, and/or HPRT RNA in the test sample is at least 1.5 times greater than the level of expression of SLC9A3R1 in the control sample.

50. The method of claim 40, wherein the test sample is isolated from a tissue of a patient suffering from ovarian cancer, breast cancer, lung cancer, prostate cancer, non-small cell lung carcinoma, and colon cancer.

51. A kit for diagnosing or detecting neoplasia, comprising:
a) a first probe specific for the detection of SLC9A3R1; and
b) a second probe specific for the detection of a neoplasia marker selected from the group consisting of CRAB-PII, enolase I, cytokeratine 18, triosephosphate isomerase, SFN, and/or HPRT.

52. The kit of claim 51, wherein the probe for detecting SLC9A3R1 is an anti-SLC9A3R1 antibody or an SLC9A3R1-binding fragment thereof.

53. The kit of claim 51, wherein the probe for detecting SLC9A3R1 is an aptamer, SLC9A3R1 ligand, or SLC9A3R1 inhibitor.

54. The kit of claim 51, wherein the second probe is selected from the group consisting of a CRAB-PII RNA binding agent, a cytokeratin 18 RNA binding agent, a triosephosphate Isomerase, binding agent, a SFN RNA binding agent, HPRT binding agent, a enolase I binding agent, and combinations thereof.

55. The kit of claim 51, further comprising a solid support to which the first and/or second probes is/are immobilized or can be immobilized.

56. The kit of claim 51, wherein the SLC9A3R1 probe is an SLC9A3R1-specific nucleic acid probe selected from the group consisting of RNA, cDNA, cRNA, and RNA-DNA hybrids.

57. The kit of claim 56, wherein the SLC9A3R1 probe is complementary to at least a 20 nucleotides of a nucleic acid sequence consisting of SEQ ID NO: 1.

58. The kit of claim 54, wherein the second probe is an SLC9A3R1-specific nucleic acid probe selected from the group consisting of RNA, cDNA, cRNA, and RNA-DNA hybrids.

59. The kit of claim 58 wherein the second probe is a nucleic acid probe complementary to at least a 20 nucleotide sequence of a nucleic acid sequence selected from the group consisting of SEQ ID NOS: 2, 3, 4, 5, 6, and 7.

60. The kit of claim 51, wherein the first probe binds to an anti-SLC9A3R1 antibody.

61. The kit of claim 51, wherein the first probe and the second probe further comprises a detectable label.
